(19) United States
(12) Patent Application Publication
(10) Pub. No.: US 2025/0170242 A1
(43) Pub. Date: May 29, 2025

(54) CHIMERIC LACTATE RECEPTOR ENGINEERED T CELLS

(71) Applicant: University of Florida Research Foundation, Incorporated, Gainesville, FL (US)

(72) Inventors: Loic Deleyrolle, Gainesville, FL (US); Duane Mitchell, Gainesville, FL (US); Jianping Huang, Gainesville, FL (US); Linchun Jin, Gainesville, FL (US)

(21) Appl. No.: 18/842,078

(22) PCT Filed: Mar. 16, 2023
(86) PCT No.: PCT/US2023/064510

§ 371 Date: Aug. 28, 2024

Related U.S. Application Data
(60) Provisional application No. 63/320,803, filed on Mar. 17, 2022.

(51) Int. Cl. A61K 40/40; A61K 35/17; A61K 40/11; A61K 40/31; A61P 35/00; C07K 14/705; C12N 5/0783

(52) U.S. Cl. CPC A61K 40/429; A61K 35/17; A61K 40/11; A61K 40/31; A61P 35/00; C07K 14/705; C07K 14/70521; C12N 5/0636; A61K 2239/15; A61K 2239/22; A61K 2239/47; C12N 2510/00

(57) ABSTRACT

Described are chimeric lactate receptors that act a molecular switches. A chimeric lactate receptor comprises a lactate receptor linked to one or more intracellular signaling domains. Also described are nucleic acids encoding the chimeric lactate receptors, T cell expressing the chimeric lactate receptors, and method of using the T cells to treat cancer.

### CROSS-REFERENCE TO RELATED APPLICATIONS

This application claims the benefit of U.S. Provisional Application No. 63/320,803, filed Mar. 17, 2022, which is incorporated herein by reference.

### SEQUENCE LISTING

The Sequence Listing written in file 590739_SeqListin_ST26.xml is 15 kilobytes in size, was created Mar. 16, 2023, and is hereby incorporated by reference.

### BACKGROUND

T cell therapies represent promising methods to treat cancer. However, these approaches face many challenges in solid tumors such as high-grade glioma (HGG). One of the major obstacles is immunosuppression caused by the tumor metabolic landscape. Tumors reprogram their metabolism, including increasing glycolytic flux. Enhanced tumor glucose uptake activity leads to a glucose restricted tumor microenvironment. Glucose restriction results in downregulation and internalization of glucose transporters (e.g., GLUT1) by T cells, which further limits glucose access, leading to T cell impairment. Additionally, cancer cell metabolic rewiring releases increased amounts of lactate, which is a potent inhibitory regulator of T cells. Due to this altered cellular metabolism, tumors can impose a metabolic imbalance restricting the therapeutic effect of T cell therapies.

### SUMMARY

Described are compounds and compositions, and methods of using the compounds and compositions for engineering cells suitable for use in T cell therapy. The engineered T cells use lactate signaling to enhance T cell glycolysis, activation, and tumor targeting.

Described are chimeric lactate receptors that act a molecular switches. A chimeric lactate receptor comprises a lactate receptor domain linked to one or more intracellular signaling domains. The lactate receptor domain can be, but is not limited to, LacR or a lactate binding fragment thereof. The intracellular signaling domain generates a signal that promotes proliferation, activation, differentiation, or an immune function in a cell expressing the chimeric lactate receptor in response to binding of lactate by the lactate receptor domain.

The intracellular signaling domain can be a intracellular signaling domain known to function in chimeric T cell receptors. The signaling domain can derived from a primary signaling domain or a costimulatory protein signaling domain. In some embodiments, a signaling domain comprises a immunoreceptor tyrosine-based activation motif (ITAM). The intracellular signaling domain can be, but is not limited to, a CD28 domain, a CD3-zeta domain, a 4-1BB domain, an OX-40 domain, a CD27 domain, a DAP10 domain, an inducible costimulatory (ICOS) domain, a 2B4 domain, an AKT domain, an IRS domain, or a PI3K domain. The chimeric lactate receptor may contain a single intracellular signaling domain or include two or more intracellular signaling domains. The chimeric lactate receptor may contain portions of one, two, or more intracellular signaling domains. A chimeric lactate receptor having two or more intracellular signaling domains can have two of the same intracellular signaling domain (e.g., two CD28 domains) or two different intracellular signaling domains (e.g., a CD28 domain and CD3-zeta domain). Binding of a lactate to the lactate receptor domain results in the intracellular signaling domain delivering a signal to, or activating, the T cell.

In some embodiments, a chimeric lactate receptor comprises LacR, or a lactate binding fragment thereof, and the signaling domain comprises an intracellular domain of CD28. In some embodiments, the LacR comprises a full length LacR or lactate binding region and transmembrane region of LacR.

Described are engineered T cells expressing one or more of the described chimeric lactate receptors. The engineered T cells are functional and resistant to the inhibitory effect of lactate. The engineered T cells can engage the glycolytic and CD28 activation pathways in response to lactate. The engineered T cells can be activated in response to lactate present in a tumor or tumor microenvironment.

Described are engineered T cells overexpressing a lactate receptor. The lactate receptor can be an endogenous lactate receptor or a heterologous lactate receptor. In some embodiments, a lactate receptor comprises LacR or a lactate binding fragment thereof. In some embodiments, the LacR comprises a full length LacR or the lactate binding region and transmembrane region of LacR. The engineered T cells are functional and resistant to the inhibitory effects of lactate. The engineered T cells can engage the glycolytic and CD28 activation pathways in response to lactate. The engineered T cells can be activated in response to lactate present in a tumor or tumor microenvironment.

Also described are nucleic acids encoding the chimeric lactate receptors or heterologous lactate receptors. The nucleic acid can be an RNA or a DNA.

In some embodiments, the engineered T cells can be used to treat cancer. The cancer can be, but is not limited to, brain tumor, such as high-grade glioma. Methods of treating cancer using the engineered T cells are described.

### BRIEF DESCRIPTION OF THE FIGURES

FIG. 1. Diagram illustrating an example of metabolic competition in the tumor microenvironment.

FIG. 2. Diagram illustrating use of a chimeric lactate receptor as a metabolic switch to overcome tumor metabolic competition.

FIG. 3A. Diagram illustrating lactate receptor, LacR-CD28 (Lac28) chimeric lactate receptor, and ΔLacR28 chimeric lactate receptor constructs. The constructs are shown with optional 2A and EGFP reporter gene components.

FIG. 3B. Gel illustrating electrophoresis of digested constructs.

FIG. 4A. Flow cytometry graphs illustrating Jurkat T-cell transduction efficiency as determined by GFP fluorescence.

FIG. 4B. Fluorescent microscopy images illustrating Jurkat T-cell transduction efficiency as determined by GFP fluorescence.

FIG. 4C. Images illustrating Lactate receptor (LacR) expression as determined by flow cytometry.

FIG. 5. Diagram and graph illustrating expression of IL-2 in T cells engineered to express LacR or LacR28 following activation with anti-CD3 antibodies.

FIG. 6A. Diagram illustrating experimental design for analyzing IL-2 expression in T cells engineered to express LacR or LacR28 in the presence of anti-CD3 antibodies and lactate.

FIG. 6B. Graph illustrating expression of IL-2 in T cells engineered to express LacR or LacR28 in the presence of anti-CD3 antibodies and lactate.

FIG. 6C. Graph and cytometry data illustrating CD25 expression in T cells engineered to express LacR or LacR28 in the presence of anti-CD3 antibodies and lactate.

FIG. 7A. Diagram illustrating experimental design for analyzing activation of T cells engineered to express LacR or LacR28 in the presence of lactate.

FIG. 7B. Graph illustrating expression of IL-2 in T cells engineered to express LacR or LacR28 in the presence of lactate.

FIG. 7C. Graphs and cytometry data illustrating CD25 and CD69 expression in T cells engineered to express LacR28 in the presence of lactate.

FIG. 7D. Graphs and cytometry data illustrating GLUT1 expression in T cells engineered to express LacR28 in the presence of lactate

FIG. 8A. Diagram illustrating an chimeric lactate receptor.

FIG. 8B. Diagram illustrating an exemplary LacR-CD28 chimeric lactate receptor.

FIG. 9A. Graphs illustrating CD69, CD25, and IL-2 expression in primary T cells expressing EGFP or LacR28 following exposure to anti-CD3 antibody plus 15 mM lactate.

FIG. 9B. Graphs illustrating CD69, CD25, and IL-2 expression in primary T cells expressing EGFP or LacR28 following exposure to plus 15 mM lactate.

### DETAILED DESCRIPTION

### A. Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a drug” includes a plurality of drugs and the like. The conjunction “or” is to be interpreted in the inclusive sense, i.e., as equivalent to “and/or,” unless the inclusive sense would be unreasonable in the context.

In general, the term “about” indicates variation in a quantity of a component of a composition not having any significant effect on the activity or stability of the composition. For example, “about” can mean within 1 standard deviation. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed. When the specification discloses a specific value for a parameter, the specification should be understood as alternatively disclosing the parameter at “about” that value. All ranges are to be interpreted as encompassing the endpoints in the absence of express exclusions, such as “not including the endpoints”; thus, for example, “within 10-15” or “from 10 to 15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of.” “Consisting essentially of” means that additional component(s), composition(s) or method step(s) that do not materially change the basic and novel characteristics of the compositions and methods described herein may be included in those compositions or methods.

A “nucleic acid” includes both RNA and DNA. RNA and DNA include, but are not limited to, cDNA, genomic DNA, plasmid DNA, synthetic RNA or DNA, and mRNA. A nucleic acid can be formulated with a delivery agent such as, but not limited, a cationic lipid, a peptide, a cationic polymer, or a virus. Nucleic acid also includes modified RNA or DNA.

An “expression vector” refers to a nucleic acid (e.g., RNA or DNA) encoding one or more expression products (e.g., peptide (i.e., polypeptide or protein)). An expression vector may be, but is not limited to, a virus or attenuated virus (viral vector), a plasmid, a linear DNA molecule, an mRNA, a CRISPR RNA, a CISPR system, or a composition comprising the nucleic acid encoding the expression product. An expression vector is capable of expressing one or more polypeptides in a cell, such a mammalian cell. The expression vector may comprise one or more sequences necessary for expression of the encoded expression product. A variety of sequences can be incorporated into an expression vector to alter expression of the coding sequence. The expression vector may comprise one or more of: a 5′ untranslated region (5′ UTR), an enhancer, a promoter, an intron, a 3′ untranslated region (3′ UTR), a terminator, and a polyA signal operably linked to the DNA coding sequence. A vector may also comprise one or more sequences that alter stability of a messenger RNA (mRNA), RNA processing, or efficiency of translation. Any of the described nucleic acids encoding a lactate receptor or a chimeric lactate receptor can be part of an expression vector designed to express the lactate receptor or chimeric lactate receptor in a cell.

A viral vector can be, but is not limited to, a pseudotyped viral vector, an AAV vector, an adenovirus, a retrovirus, a gammaretrovirus, a lentivirus, a vaccinia virus, a vesicular stomatitis virus, an alphavirus, or a herpesvirus. An adeno-associated virus can be, but is not limited to, AAV1, AAV2, AAV2/1, AAV2/2, AAV2/4, AAV2/5, AAV2/6, AAV2/7, AAV2/8, AAV2/9, AAV4, AAV5, AAV6, AAV7, AAV7m8, AAV8, AAV9, and AAV44. Nucleic acid encoding the desired protein can be packaged into the viral vectors using methods and constructs known in the art. Vectors can be manufactured in large scale quantities and/or in high yield. Vectors can be manufactured using GMP manufacturing. Vectors can be formulated to be safe and effective for injection into a mammalian subject. Vectors can be delivery to a cell, a subject, an organ or tissue in the subject, or cells in a subject using methods known in the art.

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339:823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN [A/C/T]RRT, NGAN, NGCG, NGAG, NGNG, NGC, and NGA.

A “trans-activating CRISPR RNA” (tracrRNA) is an RNA species that facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

The term “plasmid” refers to a nucleic acid that includes at least one sequence encoding a polypeptide (e.g., an expression vector) that is capable of being expressed in a mammalian cell. A plasmid can be a closed circular DNA molecule. A variety of sequences can be incorporated into a plasmid to alter expression of the coding sequence or to facilitate replication of the plasmid in a cell. Sequences can be used that influence transcription, stability of a messenger RNA (mRNA), RNA processing, or efficiency of translation. Such sequences include, but are not limited to, 5′ untranslated region (5′ UTR), promoter, introns, and 3′ untranslated region (3′ UTR). In some embodiments, plasmids can be transformed into bacteria, such as E. coli.

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter. Examples of promoters can be found, for example, in WO 2013/176772. The promoter can be, but is not limited to, CMV promoter, Igk promoter, mPGK, SV40 promoter, β-actin promoter (such as, but not limited to a human or chicken β-actin promoter), α-actin promoter, SRα promoter, herpes thymidine kinase promoter, herpes simplex virus (HSV) promoter, mouse mammary tumor virus long terminal repeat (LTR) promoter, adenovirus major late promoter (Ad MLP), rous sarcoma virus (RSV) promoter, and EFla promoter. The CMV promoter can be, but is not limited to, CMV immediate early promoter, human CMV promoter, mouse CNV promoter, and simian CMV promoter. The promoter can also be a hybrid promoter. Hybrid promoters include, but are not limited to, CAG promoter.

“Operable linkage” or being “operably linked” refers to the juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

A “heterologous” sequence is a sequence which is not normally present in a cell, genome, or gene in the genetic context in which the sequence is currently found. A heterologous sequence can be a sequence derived from the same gene and/or cell type, but introduced into the cell or a similar cell in a different context, such as on an expression vector or in a different chromosomal location or with a different promoter. A heterologous sequence can be a sequence derived from a different gene or species than a reference gene or species. A heterologous sequence can be from a homologous gene from a different species, from a different gene in the same species, or from a different gene from a different species. For example, a regulatory sequence may be heterologous in that it is linked to a different coding sequence relative to the native regulatory sequence.

The term “cancer” includes a myriad of diseases generally characterized by inappropriate cellular proliferation, abnormal or excessive cellular proliferation. Examples of cancer include, but are not limited to, breast cancer, triple negative breast cancer, colon cancer, prostate cancer, pancreatic cancer, melanoma, lung cancer, ovarian cancer, kidney cancer, brain cancer, or sarcomas.

The “tumor microenvironment” refers to the environment in and around a tumor and may include the non-malignant vascular and stromal tissue that aid in growth and/or survival of a tumor, such as by providing the tumor with oxygen, growth factors, and nutrients, or inhibiting immune response to the tumor. A tumor microenvironment includes the cellular environment in which the tumor exists, including surrounding blood vessels, immune cells, fibroblasts, bone marrow-derived inflammatory cells, lymphocytes, signaling molecules and the extracellular matrix.

A “homologous” sequence (e.g., nucleic acid sequence or amino acid sequence) refers to a sequence that is either identical or substantially similar to a known reference sequence, such that it is, for example, at least 75%, at least 80%, at least 85%, at least 90%, at least 95%, at least 96%, at least 97%, at least 98%, at least 99%, or 100% identical to the known reference sequence. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences. Homologous sequences can include, for example, orthologs (orthologous sequences) and paralogs (paralogous sequences). Homologous genes, for example, typically descend from a common ancestral DNA sequence, either through a speciation event (orthologous genes) or a genetic duplication event (paralogous genes). “Orthologous” genes are genes in different species that evolved from a common ancestral gene by speciation. Orthologs typically retain the same function in the course of evolution. “Paralogous” genes include genes related by duplication within a genome. Paralogs can evolve new functions in the course of evolution.

An “active ingredient” is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient, which is the drug substance itself, and excipients, which are the ingredients of the tablet, or the liquid in which the active agent is suspended, or other material that is pharmaceutically inert. During formulation development, the excipients can be selected so that the active ingredient can reach the target site in the body at the desired rate and extent.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein, “dose,” “unit dose,” or “dosage” can refer to physically discrete units suitable for use in a subject, each unit containing a predetermined quantity of an active pharmaceutical ingredient and/or a pharmaceutical composition thereof calculated to produce the desired response or responses in association with its administration.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those previously diagnosed with cancer. Treating can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected.

“Adoptive cell therapy,” also termed Adoptive Cell Transfer or Adoptive T Cell Transfer, is a type of immunotherapy in which T cells or engineered T cells are given to a patient to help the body fight diseases, such as cancer. In some embodiments, T cells are taken from the patient's own blood or tumor tissue, grown in large numbers in the laboratory, and then given back to the patient to help the immune system fight the cancer. In some embodiments, T cells are taken from a donor, grown in large numbers in the laboratory, and then given back to the patient to help the immune system fight the cancer. Sometimes, the T cells are changed in the laboratory to make them better able to target the patient's cancer cells and kill them. Types of adoptive cell transfer include chimeric antigen receptor T-cell (CAR T-cell) therapy and tumor-infiltrating lymphocyte (TIL) therapy. Adoptive cell transfer is also called adoptive cell therapy, cellular adoptive immunotherapy, and T-cell transfer therapy.

Chimeric antigen receptor T-cell (CAR T-cell) therapy is a type of treatment in which a patient's T cells altered (engineered) to will attack cancer cells. The T cells can be taken from a patient's or a donor's blood. A gene encoding the CAR (a receptor that binds to an antigen expressed by the patient's cancer cells) is then engineered into the T cell. This receptor is called a chimeric antigen receptor (CAR). CAR T cells can be expanded and given to the patient, such as by infusion.

A “chimeric antigen receptor” (CAR) is a synthetic receptor comprising an extracellular antigen recognition domain, an extracellular hinge region, a transmembrane domain, and an intracellular T cell signaling domain. Binding of the CAR to its cognate antigen results in the generation of an intracellular signal that activates a T cell expressing the CAR.

A “high-grade glioma” (HGG) is a type of tumor formed in the brain or spinal cord (central nervous system or CNS) through the abnormal growth of glial cells. Glial cells surround, protect, and help with the functions of neurons. Gliomas are classified into different tumor types based on which glial cells the tumor developed from, what part of the brain the tumor grows in, and how aggressive the tumor cells appear under the microscope. There are four different types of glial cells that can turn into a glial tumor: ependymal cells, microglia, astrocytes and oligodendrocytes. Then gliomas are also categorized into four different grades, based on how aggressive the tumor cells are and how fast the tumor grows. HGGs are graded as a 3 or 4, indicating they are more aggressive and grow more rapidly. HGGs have substantial morbidity and mortality. Current standard-of-care treatments available for patients with HGG include maximal surgical resection, concurrent radiotherapy and chemotherapy (i.e., temozolomide), followed by adjuvant temozolomide. Despite these aggressive interventions, the reported median survival ranges from 13 to 73 months with a 5-year survival of less than 20% in children and 5% in adults.

A defining hallmark of HGG is altered metabolism. Even though HGG exhibit a certain level of metabolic heterogeneity and adaptability, one of the dominant metabolic features of these tumors is a shift towards aerobic glycolysis, regardless of oxygen availability. This phenomenon is known as the Warburg effect. One of the strategies developed by tumor cells to meet their bioenergetic and biosynthetic demands and support their increased need for glucose is the overexpression of glucose transporters such as GLUT1 and GLUT3.

### B. Introduction

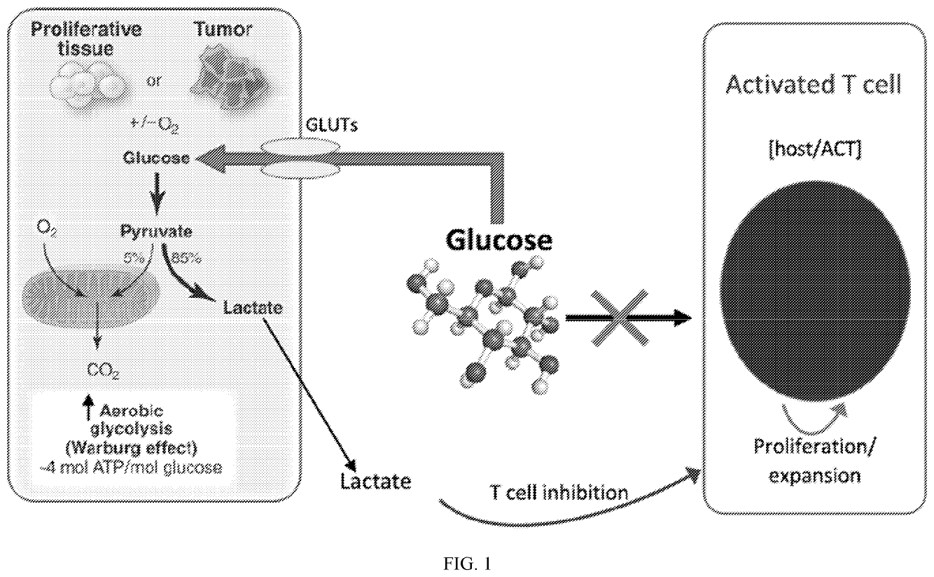

Tumor cells undergo metabolic reprogramming to enhance their glycolytic flux supporting exponential growth. A result of this tumor metabolic shift is the increase of tumor glucose uptake and utilization, depriving T cells of this nutrient which is also important to their survival, activation and expansion. Another consequence of this tumor metabolic activity is the production and secretion of large amounts of lactate into the tumor microenvironment. Lactate exerts an inhibitory effect on T cells, resulting in decreased T cell activation and contributing to tumor immune evasion. Immune evasion and metabolic reprogramming are now well recognized hallmarks of cancer and are considered to be functionally linked. The metabolic switch observed in cancer, such as HGG, leading to increased glycolysis, impacts the tumor microenvironment, which in turn acts as a barrier for successful targeting of cancer by antitumor immune cells like T cells. The increase in tumor glucose consumption imposes metabolic pressure on T cells, which experience glucose restriction (FIG. 1). This metabolic competition for glucose leads to reduced expression and membrane trafficking of GLUTs in T cells further limiting glucose access and further impairing T cell activation (Cham C M et al. “Glucose deprivation inhibits multiple key gene expression events and effector functions in CD8+ T cells.” Eur J Immunol 2008, 38:2438-2450). Glucose competition assay revealed a preferential uptake of glucose by glioma cells when co-cultured with T cells, with a decrease of glucose uptake in T cells as the number of tumor cells increased. Our data also demonstrated a positive correlation between glucose concentration and T cell activation as measured by IFNγ secretion. Together, the results indicate that increasing glucose availability for T cells to improve glycolysis represents a vial strategy to enhance T cell activation.

T cells initiate metabolic regulation to drive their activation and effector functions. Activated T cells also engage aerobic glycolysis, consuming large amounts of glucose. Blocking glycolysis reduces T cell activation. Roles for GLUT1 and GLUT3 have been demonstrated in proliferative thymocytes and T effector cells. T cells can modulate nutrient utilization, particularly glucose, and engage in metabolic regulation to sustain survival, proliferation and immune functions.

The increased glycolytic activity of cancer (e.g., HGG) cells results in increased production and secretion of lactate into the tumor microenvironment. Lactate acts as an oncometabolite and as a inhibitory regulator of T cells. Recent studies have suggested roles of lactate in the tumor microenvironment, as a metabolic fuel or as a signaling molecule regulating angiogenesis, invasion, resistance to treatments, and immunological escape (FIG. 1).

Lactate is transported by monocarboxylate transporters (MCT) into the cells, where it serves as metabolic intermediate. G protein-coupled receptor (GPCR, also known as lactate receptor, LacR, Hydroxycarboxylic acid receptor 1 (HCAR1), or G-protein-coupled receptor 81 (GPR81)) mediates lactate signaling. The effects of lactate on T cell immune function is primarily mediated through its transport by MCTs.

Physiological levels of lactic acid range from 1.5 mM to 3 mM in blood and tissues from healthy individuals. Greater concentrations are often indicative of a health problem. In cancers, lactate levels can reach up to 30 mM. In human astrocytoma, a positive correlation was found between the grade of lesions and lactate levels, which were up to 15 mM in concentration (Pucino V et al. “Lactate at the crossroads of metabolism, inflammation, and autoimmunity.” Eur J Immunol 2017, 47:14-21).

T cell activation is typically initiated by ligand (antigen) engagement with the T cell receptor (TCR). This engagement, however, is not sufficient, but itself, for the complete activation of T cells. Full activation of T cell typically which requires a secondary co-stimulatory signal. The CD28 molecule acts as a costimulatory receptor in promoting full activation of naïve T cells. CD28 co-stimulation drives intracellular biochemical events including phosphorylation and transcriptional signaling, metabolic regulation, and the production of key cytokines, chemokines, and survival factors for long-term expansion and differentiation of T cells. CD28 functions as an amplifier of TCR signaling and plays a role in many T cell processes, including delivering signals that control multiple pathways such as regulation of T cell metabolism. Specifically, the CD28 pathway increases T cell glycolytic rate, allowing to generate the energy for activation, expansion and function. It was shown that CD28 co-stimulation is important for T cell glucose uptake by up-regulating expression and promoting cell surface trafficking of GLUT1 (Jacobs S R et al. “Glucose uptake is limiting in T cell activation and requires CD28-mediated Aki-dependent and independent pathways.” J Immunol 2008, 180:4476-4486). Enhancing access to glucose, through increased level of glucose transporters and their localization at the membrane, and augmenting activity of the glycolytic pathways appears to be a mechanism by which CD28 signaling can stimulate T cell activation, growth, and sustained response.

Described are chimeric antigen receptors that take advantage of increased lactate concentration in a tumor microenvironment to increase T cell activation. The described chimeric antigen receptors combine a lactate receptor lactate binding domain and a co-stimulator to form a chimeric lactate receptor. The chimeric lactate receptor co-opt lactate to activate T cells to enhance glycolysis and activation. The chimeric antigen receptors transform lactate signaling into a signal that enhances T cell glycolysis and activation and overcomes immunosuppression driven by lactate and glucose restriction imposed by tumor cells. The co-stimulator can be, but is not limited, CD28.

### C. Chimeric Lactate Receptors

Described are chimeric lactate receptors, nucleic acids encoding the chimeric lactate receptors, and methods of using chimeric lactate receptors and nucleic acids encoding the chimeric lactate receptors to engineer cells suitable for use in T cell therapy. Expression of a chimeric antigen receptor in the engineered T cells can enhance T cell growth, glycolysis, activation, and/or tumor targeting.

A chimeric lactate receptor comprises a lactate receptor domain linked to one or more intracellular signaling domains. The lactate receptor domain can be a lactate receptor or a lactate binding fragment of a lactate receptor The intracellular signaling domain generates a signal that promotes one or more of proliferation, activation, differentiation, and an immune function in a T cell expressing the chimeric lactate receptor in response to binding of lactate by the lactate receptor domain.

The lactate receptor domain can be, but is not limited to, LacR. The lactate receptor (also known as Hydroxycarboxylic acid receptor 1 (HCA1) or G protein-coupled receptor 81 (GPR81)), is a G_{i/o}-coupled G protein-coupled receptor (GPCR) protein that in humans is encoded by the HCAR1 gene (Genbank No. NM_032554.4 (mRNA), KU285432.1 (cDNA), Gene ID: 27198, Uniprot Q9BXC0)). The primary endogenous agonist of HCA1 is lactic acid and its conjugate base, lactate. In some embodiments, the lactate binding domain comprises LacR, or a lactate binding region of LacR (SEQ ID NO: 2), e.g., ΔLacR (SEQ ID NO: 10; LacR in which the C-terminal 65 amino acids have been removed).

In some embodiments, the lactate receptor domain comprises a polypeptide having at least 70%, at least 80%, at least 90%, at least 95%, at least 97%, at least 98%, at least 99%, or 100% identity to SEQ ID NO: 2 or SEQ ID NO: 10, or an ortholog thereof, wherein the polypeptide retains the lactate binding of LacR. In some embodiments, the lactate receptor domain comprises the amino acid sequence of SEQ ID NO: 2. In some embodiments, the lactate receptor domain comprises the amino acid sequence of SEQ ID NO: 10.

The intracellular signaling domain comprises a protein known to generate a T cell activating signal or a fragment thereof capable of generating a T cell activating signal. In some embodiments, the intracellular signaling domain can be a intracellular signaling domain known in the art to function in a chimeric T cell receptor (CAR). The signaling domain can derived from a primary signaling domain (producing an activating signal 1) or a costimulatory protein signaling domain (producing an co-stimulatory signal 2). In some embodiments, a signaling domain comprises a immunoreceptor tyrosine-based activation motif (ITAM). The intracellular signaling domain can be, but is not limited to, a CD28 domain, a CD3-zeta domain, a FcεR1-γ domain, a 4-1BB domain, an OX-40 domain, a CD27 domain, a DAP10 domain, an inducible costimulatory (ICOS) domain, a 2B4 domain, an AKT domain, an IRS domain, or a PI3K domain. The chimeric lactate receptor may contain a single intracellular signaling domain or it may contain two or more intracellular signaling domains. The chimeric lactate receptor may contain portions of one, two, or more intracellular signaling domains. A chimeric lactate receptor having two or more intracellular signaling domains can have two of the same intracellular signaling domain (e.g., two CD28 domains) or two different intracellular signaling domains (e.g., a CD28 domain and CD3-zeta domain or a CD28 domain and a 4-1BB domain or OX-40 domain). A chimeric lactate receptor having two or more intracellular signaling domains can have a primary signaling domain and a co-stimulatory signaling domain or two co-stimulatory signaling domains. Binding of lactate to the lactate receptor domain results in the intracellular signaling domain delivering a signal to, or activating, the T cell.

In some embodiments, the intracellular signaling domain is a CD28 signaling domain. In some embodiments, the intracellular signaling domain comprises a polypeptide having at least, at least 80%, at least 90%, at least 95%, at least 97%, at least 98%, at least 99%, or 100% identity to SEQ ID NO: 8, wherein the polypeptide retains the T cell signaling function of CD28. In some embodiments, the intracellular signaling domain comprises the amino acid sequence of SEQ ID NO: 8.

In some embodiments, a chimeric lactate receptor comprises LacR, or a lactate binding fragment thereof, and the signaling domain comprises an intracellular domain of CD28. In some embodiments, the lactate binding domain comprises a full length LacR (SEQ ID NO: 2) or an ortholog thereof or ΔLacR (SEQ ID NO: 10) or an ortholog thereof.

In some embodiments, a chimeric lactate receptor comprises a polypeptide having at least 70%, at least 80%, at least 90%, at least 95%, at least 97%, at least 98%, at least 99%, or 100% identity to SEQ ID NO: 4 or SEQ ID NO: 6, wherein the polypeptide retains the lactate binding function of LacR and the T cell signaling function of CD28. In some embodiments, the lactate receptor domain comprises the amino acid sequence of SEQ ID NO: 4 (LacR28). In some embodiments, the lactate receptor domain comprises the amino acid sequence of SEQ ID NO: 6 (ΔLacR28).

The described chimeric lactate receptors co-opt lactate to activate T cells to enhance glycolysis and/or activation. The chimeric lactate receptor provides a metabolic switch receptor activating T cells in a tumor or tumor microenvironment having increased levels of lactate. The described chimeric lactate receptors transforming lactate signaling into a T cell activating signal to enhance T cell glycolysis and/or activation, thereby reducing immunosuppression driven by lactate and glucose restriction imposed by tumor cells.

The described chimeric lactate receptors, such as LacR28, turn a inhibitory signal, lactate, into positive signal for T cell activation and/or glycolysis. In some embodiments, the described chimeric lactate receptors increase T cell competitiveness for glucose. The described chimeric lactate receptors enhance T cell activation and function thereby facilitating the T cell in overcoming tumor-imposed immunosuppressive metabolic pressure.

In some embodiments, a T cell is engineered to overexpress a lactate receptor, or a functional fragment thereof. Overexpression of a lactate receptor or functional fragment thereof, in a T cell can turn a typically inhibitory signal, lactate, into a positive secondary signal to induce T cell activation and/or glycolysis. In some embodiments, overexpression of a lactate receptor in a T cell increases competitiveness for glucose. Overexpression of a lactate receptors in a T cell enhances T cell activation and function thereby facilitating the T cells in overcoming tumor-imposed immunosuppressive metabolic pressure.

Expression of the described chimeric lactate receptors in T cells can result in increased expression of GLUT1 in response to the presence of lactate, such as in a tumor microenvironment. Increased expression of GLUT1 can lead to increased fitness of the engineered to cell in a tumor microenvironment.

### D. Nucleic Acids Encoding Chimeric Lactate Receptors

T cells are modified to produce chimeric lactate receptor expressing T cells by introducing into a T cell a heterologous nucleic acid sequence encoding and expressing a chimeric lactate receptor.

T cells are modified to overexpress a lactate receptor by introducing into a T cell a heterologous nucleic acid sequence encoding and expressing the lactate receptor into the T cell.

A heterologous chimeric lactate receptor nucleic acid may be a recombinant nucleic acid. The nucleic acid encoding the chimeric lactate receptor coding sequence can comprise one or more regulatory sequences to facilitate expression of the chimeric lactate receptor in the T cell. Regulatory sequences include, but are not limited to, enhancers, promoters, transcription initiation sequences, translation initiation sequences, termination codons, 5′ untranslated regions (UTR) sequences, 3′ UTR sequences, transcription terminal signals, and polyA sequences. The chimeric lactate receptor coding sequence can be operably linked to a native regulatory sequence or one or more heterologous regulatory sequences. The promoter can be any promoter that is active in T cells. The promoter can be a native T cell promoter or a heterologous promoter. The promoter can be a non-viral promoter or a viral promoter. Exemplary viral promoters include, but are not limited to, a cytomegalovirus (CMV) promoter, an SV40 promoter, an RSV promoter, or a promoter found in the long-terminal repeat of the murine stem cell virus. The promoter can be a constitutive promoter or an inducible promoter.

A heterologous lactate receptor nucleic acid may be a recombinant nucleic acid. The nucleic acid encoding the lactate receptor coding sequence can comprise one or more regulatory sequences to facilitate overexpression of the lactate receptor in the T cell. Regulatory sequences include, but are not limited to, enhancers, promoters, transcription initiation sequences, translation initiation sequences, termination codons, 5′ untranslated regions (UTR) sequences, 3′ UTR sequences, transcription terminal signals, and polyA sequences. The lactate receptor coding sequence can be operably linked to a native regulatory sequence or one or more heterologous regulatory sequences. The promoter can be any promoter that is active in T cells. The promoter can be a native T cell promoter or a heterologous promoter. The promoter can be a non-viral promoter or a viral promoter. Exemplary viral promoters include, but are not limited to, a cytomegalovirus (CMV) promoter, an SV40 promoter, an RSV promoter, or a promoter found in the long-terminal repeat of the murine stem cell virus. The promoter can be a constitutive promoter or an inducible promoter.

Exemplary lactate receptor and chimeric lactate receptor amino acid and nucleic acid sequences are shown below.

LacR nucleic acid sequence
(SEQ ID NO: 1)
atgggatacaacgggtcgtgctgccgcatcgagggggacaccatctcccaggtgatgccgccgc
tgctcattgtggcctttgtgctgggcgcactaggcaatggggtcgccctgtgtggtttctgctt
ccacatgaagacctggaagcccagcactgtttaccttttcaatttggccgtggctgatttcctc
cttatgatctgcctgccttttcggacagactattacctcagacgtagacactgggcttttgggg
acattccctgccgagtggggctcttcacgttggccatgaacagggccgggagcatcgtgttcct
tacggtggtggctgcggacaggtatttcaaagtggtccacccccaccacgcggtgaacactatc
tccacccgggtggcggctggcatcgtctgcaccctgtgggccctggtcatcctgggaacagtgt
atcttttgctggagaaccatctctgcgtgcaagagacggccgtctcctgtgagagcttcatcat
ggagtcggccaatggctggcatgacatcatgttccagctggagttctttatgcccctcggcatc
atcttattttgctccttcaagattgtttggagcctgaggcggaggcagcagctggccagacagg
ctcggatgaagaaggcgacccggttcatcatggtggtggcaattgtgttcatcacatgctacct
gcccagcgtgtctgctagactctatttcctctggacggtgccctcgagtgcctgcgatccctct
gtccacggggccctgcacataaccctcagcttcacctacatgaacagcatgctggatcccctgg
tgtattatttttcaagcccctcctttcccaaattctacaacaagctcaaaatctgcagtctgaa
acccaagcagccaggacactcaaaaacacaaaggccggaagagatgccaatttcaaacctcggt
cgcaggagttgcatcagtgtggcaaatagtttccaaagccagtctgatgggcaatgggatcccc
acattgttgagtggcac
LacR amino acid sequence
(SEQ ID NO: 2)
MGYNGSCCRIEGDTISQVMPPLLIVAFVLGALGNGVALCGFCFHMKTWKPSTVYLFNLAVADFL
LMICLPFRTDYYLRRRHWAFGDIPCRVGLFTLAMNRAGSIVFLTVVAADRYFKVVHPHHAVNTI
STRVAAGIVCTLWALVILGTVYLLLENHLCVQETAVSCESFIMESANGWHDIMFQLEFFMPLGI
ILFCSFKIVWSLRRRQQLARQARMKKATRFIMVVAIVFITCYLPSVSARLYFLWTVPSSACDPS
VHGALHITLSFTYMNSMLDPLVYYFSSPSFPKFYNKLKICSLKPKQPGHSKTQRPEEMPISNLG
RRSCISVANSFQSQSDGQWDPHIVEWH
LacR28 nucleic acid sequence
(SEQ ID NO: 3)
atgggatacaacgggtcgtgctgccgcatcgagggggacaccatctcccaggtgatgccgccgc
tgctcattgtggcctttgtgctgggcgcactaggcaatggggtcgccctgtgtggtttctgctt
ccacatgaagacctggaagcccagcactgtttaccttttcaatttggccgtggctgatttcctc
cttatgatctgcctgccttttcggacagactattacctcagacgtagacactgggcttttgggg
acattccctgccgagtggggctcttcacgttggccatgaacagggccgggagcatcgtgttcct
tacggtggtggctgcggacaggtatttcaaagtggtccacccccaccacgcggtgaacactatc
tccacccgggtggcggctggcatcgtctgcaccctgtgggccctggtcatcctgggaacagtgt
atcttttgctggagaaccatctctgcgtgcaagagacggccgtctcctgtgagagcttcatcat
ggagtcggccaatggctggcatgacatcatgttccagctggagttctttatgcccctcggcatc
atcttattttgctccttcaagattgtttggagcctgaggcggaggcagcagctggccagacagg
ctcggatgaagaaggcgacccggttcatcatggtggtggcaattgtgttcatcacatgctacct
gcccagcgtgtctgctagactctatttcctctggacggtgccctcgagtgcctgcgatccctct
gtccacggggccctgcacataaccctcagcttcacctacatgaacagcatgctggatcccctgg
tgtattatttttcaagcccctcctttcccaaattctacaacaagctcaaaatctgcagtctgaa
acccaagcagccaggacactcaaaaacacaaaggccggaagagatgccaatttcaaacctcggt
cgcaggagttgcatcagtgtggcaaatagtttccaaagccagtctgatgggcaatgggatcccc
acattgttgagtggcacaggagtaagaggagcaggctcctgcacagtgactacatgaacatgac
tccccgccgccccgggcccacccgcaagcattaccagccctatgccccaccacgcgacttcgca
gcctatcgctcc
LacR28 amino acid sequence
(SEQ ID NO: 4)
MGYNGSCCRIEGDTISQVMPPLLIVAFVLGALGNGVALCGFCFHMKTWKPSTVYLFNLAVADFL
LMICLPFRTDYYLRRRHWAFGDIPCRVGLFTLAMNRAGSIVFLTVVAADRYFKVVHPHHAVNTI
STRVAAGIVCTLWALVILGTVYLLLENHLCVQETAVSCESFIMESANGWHDIMFQLEFFMPLGI
ILFCSFKIVWSLRRRQQLARQARMKKATRFIMVVAIVFITCYLPSVSARLYFLWTVPSSACDPS
VHGALHITLSFTYMNSMLDPLVYYFSSPSFPKFYNKLKICSLKPKQPGHSKTQRPEEMPISNLG
RRSCISVANSFQSQSDGQWDPHIVEWHRSKRSRLLHSDYMNMTPRRPGPTRKHYQPYAPPRDFA
AYRS
ΔLacR28 nucleic acid sequence
(SEQ ID NO: 5)
atgggatacaacgggtcgtgctgccgcatcgagggggacaccatctcccaggtgatgccgccgc
tgctcattgtggcctttgtgctgggcgcactaggcaatggggtcgccctgtgtggtttctgctt
ccacatgaagacctggaagcccagcactgtttaccttttcaatttggccgtggctgatttcctc
cttatgatctgcctgccttttcggacagactattacctcagacgtagacactgggcttttgggg
acattccctgccgagtggggctcttcacgttggccatgaacagggccgggagcatcgtgttcct
tacggtggtggctgcggacaggtatttcaaagtggtccacccccaccacgcggtgaacactatc
tccacccgggtggcggctggcatcgtctgcaccctgtgggccctggtcatcctgggaacagtgt
atcttttgctggagaaccatctctgcgtgcaagagacggccgtctcctgtgagagcttcatcat
ggagtcggccaatggctggcatgacatcatgttccagctggagttctttatgcccctcggcatc
atcttattttgctccttcaagattgtttggagcctgaggcggaggcagcagctggccagacagg
ctcggatgaagaaggcgacccggttcatcatggtggtggcaattgtgttcatcacatgctacct
gcccagcgtgtctgctagactctatttcctctggacggtgccctcgagtgcctgcgatccctct
gtccacggggccctgcacataaccctcagcttcacctacatgaacagcatgctggatcccctgg
tgtattatttttcaaggagtaagaggagcaggctcctgcacagtgactacatgaacatgactcc
ccgccgccccgggcccacccgcaagcattaccagccctatgccccaccacgcgacttcgcagcc
tatcgctcc
ΔLacR28 amino acid sequence
(SEQ ID NO: 6)
MGYNGSCCRIEGDTISQVMPPLLIVAFVLGALGNGVALCGFCFHMKTWKPSTVYLFNLAVADFL
LMICLPFRIDYYLRRRHWAFGDIPCRVGLFTLAMNRAGSIVFLTVVAADRYFKVVHPHHAVNTI
STRVAAGIVCTLWALVILGTVYLLLENHLCVQETAVSCESFIMESANGWHDIMFQLEFFMPLGI
ILFCSFKIVWSLRRRQQLARQARMKKATRFIMVVAIVFITCYLPSVSARLYFLWTVPSSACDPS
VHGALHITLSFTYMNSMLDPLVYYFSRSKRSRLLHSDYMNMTPRRPGPTRKHYQPYAPPRDFAA
YRS
CD28 signaling domain nucleic acid sequence
(SEQ ID NO: 7)
aggagtaagaggagcaggctcctgcacagtgactacatgaacatgactccccgccgccccgggc
ccacccgcaagcattaccagccctatgccccaccacgcgacttcgcagcctatcgctcc
CD28 signaling domain nucleic acid sequence
(SEQ ID NO: 8)
RSKRSRLLHSDYMNMTPRRPGPTRKHYQPYAPPRDFAAYRS
ΔLacR nucleic acid sequence
(SEQ ID NO: 9)
atgggatacaacgggtcgtgctgccgcatcgagggggacaccatctcccaggtgatgccgccgc
tgctcattgtggcctttgtgctgggcgcactaggcaatggggtcgccctgtgtggtttctgctt
ccacatgaagacctggaagcccagcactgtttaccttttcaatttggccgtggctgatttcctc
cttatgatctgcctgccttttcggacagactattacctcagacgtagacactgggcttttgggg
acattccctgccgagtggggctcttcacgttggccatgaacagggccgggagcatcgtgttcct
tacggtggtggctgcggacaggtatttcaaagtggtccacccccaccacgcggtgaacactatc
tccacccgggtggcggctggcatcgtctgcaccctgtgggccctggtcatcctgggaacagtgt
atcttttgctggagaaccatctctgcgtgcaagagacggccgtctcctgtgagagcttcatcat
ggagtcggccaatggctggcatgacatcatgttccagctggagttctttatgcccctcggcatc
atcttattttgctccttcaagattgtttggagcctgaggcggaggcagcagctggccagacagg
ctcggatgaagaaggcgacccggttcatcatggtggtggcaattgtgttcatcacatgctacct
gcccagcgtgtctgctagactctatttcctctggacggtgccctcgagtgcctgcgatccctct
gtccacggggccctgcacataaccctcagcttcacctacatgaacagcatgctggatcccctgg
tgtattatttttca
ΔLacR amino acid sequence
(SEQ ID NO: 10)
MGYNGSCCRIEGDTISQVMPPLLIVAFVLGALGNGVALCGFCFHMKTWKPSTVYLFNLAVADFL
LMICLPFRTDYYLRRRHWAFGDIPCRVGLFTLAMNRAGSIVFLTVVAADRYFKVVHPHHAVNTI
STRVAAGIVCTLWALVILGTVYLLLENHLCVQETAVSCESFIMESANGWHDIMFQLEFEMPLGI
ILFCSFKIVWSLRRRQQLARQARMKKATRFIMVVAIVFITCYLPSVSARLYFLWTVPSSACDPS
VHGALHITLSFTYMNSMLDPLVYYFS

In some embodiments, a chimeric lactate receptor nucleic acid encodes a lactate binding domain polypeptide having an amino acid sequence that has at least 70%, at least 80%, at least 90%, at least 95%, at least 97%, at least 98%, at least 99% identity to SEQ ID NO: 2, wherein the encoded polypeptide binds lactate. In some embodiments, a chimeric lactate receptor nucleic acid encodes a lactate binding domain polypeptide having the amino acid sequence of SEQ ID NO: 2. In some embodiments, a chimeric lactate receptor nucleic acid comprises a sequence having at least 70%, at least 80%, at least 90%, at least 95%, at least 97%, at least 98%, at least 99% identity to SEQ ID NO: 1, wherein the nucleic acid encodes a polypeptide that binds lactate. In some embodiments, a chimeric lactate receptor nucleic acid comprises the nucleic acid sequence of SEQ ID NO: 1.

In some embodiments, a chimeric lactate receptor nucleic acid encodes a lactate binding domain polypeptide having an amino acid sequence that has at least 70%, at least 80%, at least 90%, at least 95%, at least 97%, at least 98%, at least 99% identity to SEQ ID NO: 10, wherein the encoded polypeptide binds lactate. In some embodiments, a chimeric lactate receptor nucleic acid encodes a lactate binding domain polypeptide having the amino acid sequence of SEQ ID NO: 10. In some embodiments, a chimeric lactate receptor nucleic acid comprises a sequence having at least 70%, at least 80%, at least 90%, at least 95%, at least 97%, at least 98%, at least 99% identity to SEQ ID NO: 9, wherein the nucleic acid encodes a polypeptide that binds lactate. In some embodiments, a chimeric lactate receptor nucleic acid comprises the nucleic acid sequence of SEQ ID NO: 9.

In some embodiments, a chimeric lactate receptor nucleic acid encodes a CD28 signaling domain having an amino acid sequence that has at least 70%, at least 80%, at least 90%, at least 95%, at least 97%, at least 98%, at least 99% identity to SEQ ID NO: 8, wherein the encoded polypeptide is capable of provide a T cell activating signal. In some embodiments, a chimeric lactate receptor nucleic acid encodes a CD28 signaling domain having the amino acid sequence of SEQ ID NO: 8. In some embodiments, a chimeric lactate receptor nucleic acid comprises a sequence having at least 70%, at least 80%, at least 90%, at least 95%, at least 97%, at least 98%, at least 99% identity to SEQ ID NO: 7, wherein the nucleic acid encodes a CD28 signaling domain capable of provide a T cell activating signal. In some embodiments, a chimeric lactate receptor nucleic acid comprises the nucleic acid sequence of SEQ ID NO: 7.

In some embodiments, a chimeric lactate receptor nucleic acid encodes a chimeric lactate receptor having an amino acid sequence that has at least 70%, at least 80%, at least 90%, at least 95%, at least 97%, at least 98%, at least 99% identity to SEQ ID NO: 4, wherein the encoded polypeptide is capable of binging lactate and providing a T cell activating signal. In some embodiments, a chimeric lactate receptor nucleic acid encodes a polypeptide having the amino acid sequence of SEQ ID NO: 4. In some embodiments, a chimeric lactate receptor nucleic acid comprises a sequence having at least 70%, at least 80%, at least 90%, at least 95%, at least 97%, at least 98%, at least 99% identity to SEQ ID NO: 3, wherein the nucleic acid encodes a chimeric lactate receptor capable binding lactate and providing a T cell activating signal. In some embodiments, a chimeric lactate receptor nucleic acid comprises the nucleic acid sequence of SEQ ID NO: 3.

In some embodiments, a chimeric lactate receptor nucleic acid encodes a chimeric lactate receptor having an amino acid sequence that has at least 70%, at least 80%, at least 90%, at least 95%, at least 97%, at least 98%, at least 99% identity to SEQ ID NO: 6, wherein the encoded polypeptide is capable of binging lactate and providing a T cell activating signal. In some embodiments, a chimeric lactate receptor nucleic acid encodes a polypeptide having the amino acid sequence of SEQ ID NO: 6. In some embodiments, a chimeric lactate receptor nucleic acid comprises a sequence having at least 70%, at least 80%, at least 90%, at least 95%, at least 97%, at least 98%, at least 99% identity to SEQ ID NO: 5, wherein the nucleic acid encodes a chimeric lactate receptor capable binding lactate and providing a T cell activating signal. In some embodiments, a chimeric lactate receptor nucleic acid comprises the nucleic acid sequence of SEQ ID NO: 5.

The chimeric lactate receptor nucleic acid can be, but is not limited to, a DNA, an RNA, an mRNA, a double stranded nucleic acid, a single stranded nucleic acid, a plasmid, an expression vector, a viral vector, or a CRISPR construct. One or more nucleotides or nucleobases in the nucleic acid may be modified. Viral vectors include, but are not limited to, retroviral vectors, gammaretrovirus, alphavirus, adenovirus, adeno-associated virus, vaccinia virus, herpes virus, and lentivirus. A viral vector can be engineered to transform T cells.

### E. Chimeric Lactate Receptor T Cells

Described are chimeric lactate receptor expressing T cells, Also described are compositions, including pharmaceutical compositions, containing the chimeric lactate receptor expressing T cells. Also described are formulations comprising the chimeric lactate receptor expressing T cells. Chimeric lactate receptor expressing T cells and compositions and formulations comprising the chimeric lactate receptor expressing T cells can be used in Adoptive T Cell Transfer, such as to treat cancer. Methods of using the chimeric lactate receptor expressing T cells and compositions and formulations comprising the chimeric lactate receptor expressing T cells to treat a subject having a cancerous tumor, such as a solid cancerous tumor are also described.

In some embodiment, a chimeric lactate receptor expressing T cell comprises a heterologous nucleic acid sequence that provides for expression of a chimeric lactate receptor.

Described are lactate receptor overexpressing T cells, compositions, including pharmaceutical compositions, comprising the chimeric lactate receptor expressing T cells, and formulations comprising the lactate receptor overexpressing T cells. Lactate receptor overexpressing T cells and compositions and formulations comprising the lactate receptor overexpressing T cells can be used in Adoptive T Cell Transfer, such as to treat cancer. Methods of using the lactate receptor overexpressing T cells and compositions and formulations comprising the lactate receptor overexpressing T cells to treat a subject having a cancerous tumor, such as a solid cancerous tumor are also described

In some embodiment, a lactate receptor overexpressing T cell comprises a T cell having a heterologous nucleic acid sequence the provides for expression of a lactate receptor in the T cell.

Described are methods of modifying T cells comprising introducing into one or more T cells, an expression vector encoding a lactate receptor or chimeric lactate receptor, wherein introducing the expression vector encoding the lactate receptor or chimeric lactate receptor into the T cell results in expression of the lactate receptor or chimeric lactate receptor by the T cell.

The T cell can be any T cell. The T cell may be, but is not limited to, a cultured T cell, a primary T cell, a T cell from a cultured T cell line, or a T cell obtained from a mammal. A T cell from a cultured T cell line includes, but is not limited to, a Jurkat T cell and a SupT1. A primary T cell can be from the subject (autologous T cell) to be treated or a donor subject (allogeneic T cell).

The T cell can be sourced from a mammal. The T cell can be, but is not limited to, a T cell obtained from blood, a T cell obtained from bone marrow, a T cell obtained from a lymph node, a T cell obtained from a thymus, a tumor infiltrating lymphocyte (TIL), a T cell obtained from a spleen, or a T cell from umbilical cord blood, each of which can be from an autologous donor source or an allogeneic donor source. The T cell can also be a universal allogenic T cell (e.g., induced pluripotent stem cells (iPSCs), HSCs) or a universal CAR T cell. In some embodiments, the T cell is a human T cell. In some embodiments, the T cell is obtained from a human subject.

The T cell can be any type of T cell. The T cell can, but is not limited to, a naïve T cell (T_{naïve} cell), an effector T cell, an effector memory T cell (Tₑₘ cell), a CD4+/CD8+ T cell, a helper T cell, a CD4+ T cell, a CD4+ helper T cell, a Th1 T cell, a Th2 T cell, a cytotoxic T cell, a CD8+ T cell, peripheral blood mononuclear cell (PBMC), a peripheral blood leukocyte (PBL), a tumor infiltrating T cell (TIL), a memory T cell, a central memory T cell (T_{cm} cell), a regulatory T cell, an αβ T cell, a γδ T cell, a modified T cell, a T cell for use in adoptive cell transfer therapy (e.g., adoptive T cell transfer therapy), a TCR-engineered T cell, a chimeric antigen receptor (CAR) T cell, a first generation CAR T cell (CAR having a single signaling domain, such as a CD3ζ signaling domain), a second generation CAR T cell (CAR having a co-stimulatory domain), a third generation CAR T cell (CAR having multiple co-stimulatory domains), a fourth generation CAR T cell (TRUCK or armored CAR), dual-antigen receptor CAR T cell, or a CAR T cell having an inducible suicide gene, or a combination thereof. In some embodiments, the T cell has been previously modified, e.g., a CAR T cell. The T cell can be a single T cell or a population of T cells. In some embodiments, the population of T cells is a substantially homogenous population with respect to the type of T cell (e.g., the population of T cells may be CD4⁺/CD8⁺ T cells, cytotoxic T cells, or CAR T cells). In some embodiments, the population of T cells is a heterogenous population with respect to the type of T cell (e.g., comprising cytotoxic T cells and helper T cells). In some embodiments, the population of T cells is an essentially clonal population of T cells.

The T cell may be isolated or purified from a source using any suitable technique known in the art for isolating or purifying T cells. The T cell (population) can be expanded prior to modification to express a lactate receptor or chimeric lactate receptor, or after modification to express a lactate receptor or chimeric lactate receptor. The T cells can be isolated, enriched, or purified prior to and/or after modification to express a lactate receptor or chimeric lactate receptor gene. Expansion of the T cell population can be done using any available method in the art for expanding T cells.

A T cell can be modified to express a chimeric lactate receptor by introducing into the T cell a nucleic acid encoding the chimeric lactate receptor. Introducing the chimeric lactate receptor nucleic acid into the T cell may be done using any method available in the art. For example, the chimeric lactate receptor nucleic acid can be introduced into a T cell by various transfection or transformation methods known in the art. Chimeric lactate receptor nucleic acid can be introduced into a T cell using a viral vector or a non-viral vector. Methods of introducing a nucleic acid into a cell include, but are not limited to, viral vectors, microinjection, microprojectile bombardment (e.g., gene gun), electroporation, lipofection, and CRISPR (e.g., CRISPR-Cas9) systems.

A T cell can be modified to overexpress a lactate receptor by introducing into the T cell a nucleic acid encoding the lactate receptor. Introducing the lactate receptor nucleic acid into the T cell may be done using any method available in the art. For example, the lactate receptor nucleic acid can be introduced into a T cell by various transfection or transformation methods known in the art. A lactate receptor nucleic acid can be introduced into a T cell using a viral vector or a non-viral vector. Methods of introducing a nucleic acid into a cell include, but are not limited to, viral vectors, microinjection, microprojectile bombardment (e.g., gene gun), electroporation, lipofection, and CRISPR (e.g., CRISPR-Cas9) systems.

In some embodiments, a marker gene, such as a GFP, is introduced with the lactate receptor or chimeric lactate receptor nucleic acid into the T cell to aid in monitoring transfection. In some embodiments, the marker gene allows for selection of transfected T cells.

In some embodiments, the lactate receptor-overexpressing or chimeric lactate receptor expressing T cell is further modified. The further modification can be done prior to modifying the T cell to express the chimeric lactate receptor or overexpress the lactate receptor, simultaneously with modifying the T cell to express the chimeric lactate receptor or overexpress the lactate receptor, after modifying the T cell to express the chimeric lactate receptor or overexpress the lactate receptor, or a combination thereof.

The further modification can add one or more desired functions to the T cell. In some embodiments, the T cell is further modified by introduction of a nucleic acid encoding an additional gene. The T cell can be further modified by introducing into the T cell, one or more nucleic acids encoding one or more of: a T cell receptor (TCR), an αβ TCR, a γδ TCR, a CAR, a first generation CAR, a second generation CAR, a third generation CAR, a fourth generation CAR, a secreted cytokine (including, but not limited to, IL-12, IL-18, IL-15, and IL-7), a cytokine receptor, a chimeric cytokine receptor, a CD40L, a4-1BBL, a dominant-negative TGF-β receptor II, a constitutively active Akt, or an antibody-like protein (including, but not limited to, a single chain variable fragment (scFv), a nanobody, or a bispecific T-cell engager (BiTE)), or combinations thereof. The T cell can be further modified by introducing in the T cell one or more of: a tumor mRNA, total tumor mRNA, slow cycling cancer cell mRNA, or cancer stem cell mRNA. In some embodiments, the T cell is modified to alter or delete one or more genes normally expressed in the T cell.

In some embodiments, the lactate receptor overexpressing or chimeric lactate receptor expressing T cell expresses, is engineered to express, or has been engineered to express an antigen-specific receptor. The antigen-specific receptor can be, but is not limited to, a T cell receptor (TCR) or a chimeric antigen receptor (CAR). The antigen-specific receptor can be an endogenous antigen-specific receptor or a heterologous or recombinant antigen-specific receptor. An antigen-specific receptor is a receptor that specifically binds to and immunologically recognizes an antigen, or an epitope thereof, such that binding of the receptor to the antigen, or the epitope thereof, elicits an immune response, such as, but not limited to, activation of the T cell. The antigen-specific receptor can be, but is not limited to, a T cell receptor (TCR) or a CAR. An antigen-specific TCR generally comprises two polypeptides a α chain and a β chain or γ chain and a δ chain. The antigen-specific receptor can be an endogenous TCR, a heterologous TCR, or a recombinant TCR. In some embodiments, the antigen-specific receptor recognizes an antigen expressed by a tumor in a subject to be treated with the lactate receptor overexpressing or chimeric lactate receptor expressing T cell. A CAR typically comprises an antigen binding domain of an antibody fused to a transmembrane and an intracellular signally domain such as the intracellular signally domain of a TCR (e.g., a CD3ζ signaling domain). In some embodiments, the TCR or CAR recognized a shared tumor antigen. In some embodiments, the CAR is modified such that it has reduced or no binding affinity for a Fc receptor.

In some embodiments, the lactate receptor overexpressing or chimeric lactate receptor expressing T cells are expanded prior to modifying the T cells to overexpress the lactate receptor or express the chimeric lactate receptor. In some embodiments, the T cells are not expanded prior to modifying the T cells to overexpress the lactate receptor or express the chimeric lactate receptor. In some embodiments, the lactate receptor overexpressing or chimeric lactate receptor expressing T cells are expanded after modifying the T cells to overexpress the lactate receptor or express the chimeric lactate receptor, but prior to administration of the lactate receptor overexpressing or chimeric lactate receptor expressing T cells to the subject. In some embodiments, lactate receptor overexpressing or chimeric lactate receptor expressing T cells are not expanded after modifying the T cells to overexpress the lactate receptor or express the chimeric lactate receptor. The expansion of the numbers of T cells can be done using any method known in the art for expanding T cell numbers.

### F. Formulation

In some embodiments, the lactate receptor overexpressing or chimeric lactate receptor expressing T cells are formulated with one or more additional pharmaceutically active ingredients.

In some embodiments, the described lactate receptor overexpressing or chimeric lactate receptor expressing T cells and/or one or more additional therapeutics are combined with one or more pharmaceutically acceptable excipients to form a pharmaceutical composition. Pharmaceutically acceptable excipients (excipients) are substances other than an active pharmaceutical ingredient (API, therapeutic product) that are intentionally included with the API (molecule). Excipients do not exert or are not intended to exert a therapeutic effect at the intended dosage. Excipients may act to a) aid in processing of the API during manufacture, b) protect, support, or enhance stability, bioavailability or subject acceptability of the API, c) assist in product identification, and/or d) enhance any other attribute of the overall safety, effectiveness, of delivery of the API during storage or use. A pharmaceutically acceptable excipient may or may not be an inert substance. Excipients include, but are not limited to: adjuvants, absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The lactate receptor overexpressing or chimeric lactate receptor expressing T cells, or pharmaceutical composition thereof, can be formulated for administration to the subject by any suitable route. Suitable routes include, but are not limited to, parenteral administration, intravenous administration, intratumoral administration, intraarterial administration, intraperitoneal administration, and injection.

The lactate receptor overexpressing or chimeric lactate receptor expressing T cell can be provided as a single isolated T cell or as a population of T cells. In some embodiments, a population of T cells comprises an essentially clonal population of T cells derived from a single lactate receptor overexpressing or chimeric lactate receptor expressing T cell. In some embodiments, the population of T cells is a substantially homogenous population of lactate receptor overexpressing or chimeric lactate receptor expressing T cells. In some embodiments, a population of T cells comprises T cells in which all or substantially all of the T cells in the population are modified to overexpress a lactate receptor or express a chimeric lactate receptor. In some embodiments, the population of T cells comprises a population of T cells in which less than all of the T cells are modified to overexpress a lactate receptor or express a chimeric lactate receptor. The percentage of lactate receptor overexpressing or chimeric lactate receptor expressing T cells in a population of T cells may be at least 10%, at least 20%, at least 30%, at least 40%, at least 50%, at least 60%, at least 70%, at least 80%, at least 90%, at least 95%, or 100%. In some embodiments, the population of T cells is a heterogenous population with respect to the type of T cell.

In some embodiments, the lactate receptor overexpressing or chimeric lactate receptor expressing T cells are provided in a population of cells that includes one or more additional cells other than T cells. The additional cell can be, but is not limited to, an antigen presenting cell, a dendritic cell, a B cell, a macrophage, a neutrophil, an erythrocyte, an endothelial cell, an epithelial cell, a parenchymal cell, or a cancer cell.

### G. Methods of Use

The potential of immunotherapy in central nervous system (CNS) malignancies has previously been thought to be futile given the immune privileged and immunosuppressive nature of the intracranial environment. However, more recently it has been demonstrated that the immune system is capable of surveying the CNS and mounting an immunological response that can be therapeutically exploited to treat brain cancer. Several immunotherapeutic approaches have been advanced for the treatment of brain tumors and are currently under evaluation in clinical trials. Adoptive cellular therapy (ACT) comprising the ex vivo expansion and intravenous transfer of autologous tumor-specific lymphocytes, has emerged as a suitable platform to target refractory neoplastic cells.

The lactate receptor overexpression T cells and chimeric lactate receptor expressing T cells are useful in the treatment of cancer. The cancer can be, but is not limited to, a single solid tumor, a hematopoietic cancer, and/or a metastatic cancer.

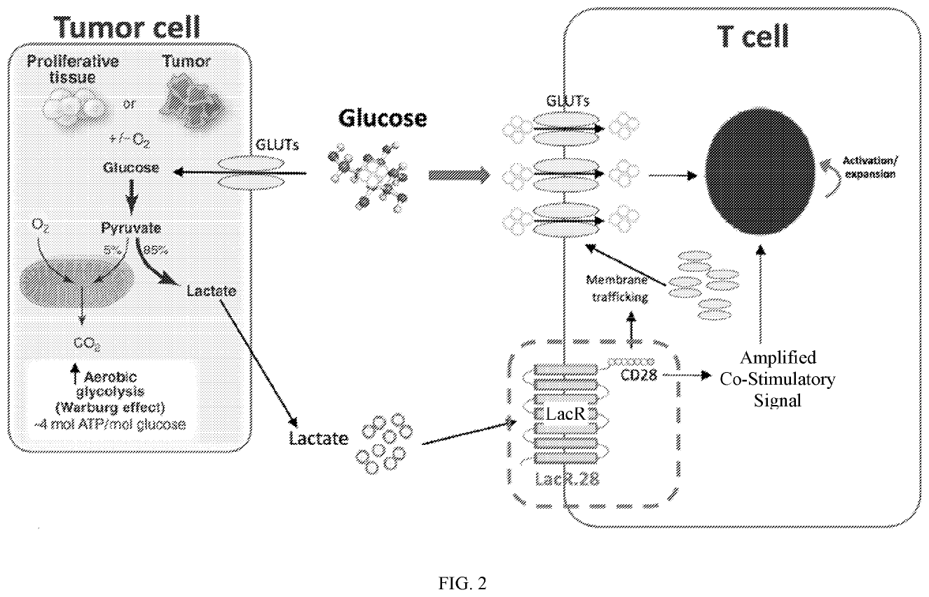

Use of engineered T cells expressing the chimeric lactate receptor to treat HGG is shown in FIG. 2. T cells engineered to overexpression a lactate receptor (or transmembrane domain thereof) can also be used.

Described are methods of using the lactate receptor overexpressing or chimeric lactate receptor expressing T cells to provide an immune response in a subject. The methods comprise administering the lactate receptor overexpressing or chimeric lactate receptor expressing T cells to a subject in need of such immune response. In some embodiments, the described methods can be used to treat cancer. In some embodiments, the described methods can be used to induce an immune response against a cancer. The methods comprises administering to subject any of the T cells described herein, or a population thereof, or a composition comprising any of the T cells described herein, in an amount effective to treat or prevent the disease in the subject.

Described are methods for treatment of a cancer in a subject comprising, administering to the subject a composition comprising an effective dose of lactate receptor overexpressing or chimeric lactate receptor expressing T cells. The methods of treating cancer include adoptive cell therapy using any of the described lactate receptor overexpressing or chimeric lactate receptor expressing T cells or pharmaceutical compositions containing the lactate receptor overexpressing or chimeric lactate receptor expressing T cells.

In some embodiments, methods of treating cancer using lactate receptor overexpressing or chimeric lactate receptor expressing T cells comprise: (a) modifying one or more T cells to form lactate receptor overexpressing or chimeric lactate receptor expressing T cells; and (b) administering the lactate receptor overexpressing or chimeric lactate receptor expressing T cells to the subject.

In some embodiments, the methods of treating cancer using lactate receptor overexpressing or chimeric lactate receptor expressing T cells comprise: (a) obtaining or having obtained one or more T cells from the subject or a donor subject; (b) modifying the T cells to form a lactate receptor overexpressing or chimeric lactate receptor expressing T cells; and (c) administering the lactate receptor overexpressing or chimeric lactate receptor expressing T cells to the subject.

In some embodiments, the methods of treating cancer using lactate receptor overexpressing or chimeric lactate receptor expressing T cells comprise: (a) obtaining or having obtained one or more T cells; (b) modifying the T cells to form lactate receptor overexpressing or chimeric lactate receptor expressing T cells; and (c) administering the lactate receptor overexpressing or chimeric lactate receptor expressing T cells to the subject.

In some embodiments, the methods of treating cancer using lactate receptor overexpressing or chimeric lactate receptor expressing T cells comprise: (a) obtaining or having obtained one or more modified T cells; (b) further modifying the modified T cells to form lactate receptor overexpressing or chimeric lactate receptor expressing T cells; and (c) administering the lactate receptor overexpressing or chimeric lactate receptor expressing T cells to the subject.

The T cell can be any T cell as described for lactate receptor overexpressing or chimeric lactate receptor expressing T cells, including, but not limited to, a T cell from a subject, a T cell from an allogeneic subject, or a cultured T cell. A cultured T cell can be a previously modified T cell. In some embodiments, the culture T cell is a CAR T cell. An allogenic source can be the same species or a different species. In some embodiments, the subject or donor subject is immunized with an antigen prior to obtaining the one or more T cells from the subject or donor subject. The antigen can be an antigen associated with the cancer to be treated in the subject.

Modifying the T cells to form lactate receptor overexpressing or chimeric lactate receptor expressing T cells comprises introducing into the T cell a heterologous lactate receptor or chimeric lactate receptor nucleic acid for overexpression of a lactate receptor or expression of a chimeric lactate receptor. In some embodiments, the heterologous chimeric lactate receptor nucleic acid comprises a chimeric lactate receptor coding sequence (e.g., SEQ ID NO: 3 or 5). In some embodiments, the heterologous chimeric lactate receptor nucleic acid comprises a sequence having at least 70%, at least 80%, at least 90%, at least 95%, at least 97%, at least 98%, at least 99%, or 100% to SEQ ID NO: 3 or 5. In some embodiments, the heterologous lactate receptor or chimeric lactate receptor nucleic acid encodes a protein having at least 70%, at least 80%, at least 90%, and least 95%, at least 97%, at least 98%, at least 99%, or 100% amino acid sequence identity to SEQ ID NO: 2 or 10, wherein the encoded polypeptide retains the lactate binding.

In some embodiments, the chimeric lactate receptor nucleic acid encodes a protein having at least 70%, at least 80%, at least 90%, and least 95%, at least 97%, at least 98%, at least 99%, or 100% amino acid sequence identity to SEQ ID NO: 4 or 6, wherein the encoded polypeptide binds lactate and provides a T cell activating signal.

The heterologous lactate receptor or chimeric lactate receptor nucleic acid encoding the lactate receptor or chimeric lactate receptor protein can be introduced into the T cell using any means available in the art. Such means include, but are not limited to, infecting the T cell with a virus or retrovirus containing the heterologous lactate receptor or chimeric lactate receptor nucleic acid, using a CRISPR-Cas system, or transfecting the cell with a DNA or RNA containing the heterologous lactate receptor or chimeric lactate receptor nucleic acid.

In some embodiments, the T cell is further modified. The further modification can be, but is not limited to, introduction in the T cell, one or more additional nucleic acids. In some embodiments, the one or more nucleic acids encode one or more of: a TCR, an αβ TCR, a γδ TCR, a CAR, a first generation CAR, a second generation CAR, a third generation CAR, a fourth generation CAR, a secreted cytokine, a cytokine receptor, a chimeric cytokine receptor, a CD40L, a 4-1BBL, a dominant-negative TGF-receptor II, a constitutively active Akt, or an antibody-like protein. In some embodiments, the one or more nucleic acids comprises: a tumor mRNA, total tumor mRNA, slow cycling cancer cell mRNA, or cancer stem cell mRNA. The T cell can be modified prior to, concurrent with, or after modifying the T cells to form lactate receptor overexpressing or chimeric lactate receptor expressing T cells.

The one or more additional nucleic acids can be introduced into the T cell using any means available in the art. Such means include, but are not limited to, infecting the T cell with a virus or retrovirus containing the one or more additional nucleic acid, using a CRISPR-Cas system, or transfecting the cell with a DNA or RNA containing the one or more additional nucleic acid.

In some embodiments, the methods comprise expanding the T cells. Expanding the T cells can be done prior to or after modifying the T cells to form lactate receptor overexpressing or chimeric lactate receptor expressing T cells. In some embodiments, the T cells are expanded prior to modifying the T cells to overexpress a lactate receptor or express a chimeric lactate receptor. In some embodiments, the T cells are not expanded prior to modifying the T cells to overexpress the lactate receptor or express the chimeric lactate receptor. In some embodiments, the T cells are expanded after modifying the T cells to overexpress the lactate receptor or express the chimeric lactate receptor, but prior to administration of the modified T cells to the subject. In some embodiments, the T cells are not expanded after modifying the T cells to overexpress the lactate receptor or express the chimeric lactate receptor. The expansion of the numbers of T cells can be done using any method known in the art for expanding T cell numbers.

In some embodiments, administering the lactate receptor overexpressing or chimeric lactate receptor expressing T cells to the subject comprises administering to the subject a population of T cells. In some embodiments, a population of T cells comprises an essentially clonal population of T cells derived from a single lactate receptor overexpressing or chimeric lactate receptor expressing T cell. In some embodiments, the population of T cells is a substantially homogenous population of lactate receptor overexpressing or chimeric lactate receptor expressing T cells. In some embodiments, a population of T cells comprises T cells in which all or substantially all of the T cells in the population are modified to overexpress of lactate receptor or express a chimeric lactate receptor. In some embodiments, the population of T cells comprises a population of T cells in which less than all of the T cells are modified to lactate receptor overexpressing or express the chimeric lactate receptor. The percentage of lactate receptor overexpressing or chimeric lactate receptor expressing T cells in a population of T cells may be at least 10%, at least 20%, at least 30%, at least 40%, at least 50%, at least 60%, at least 70%, at least 80%, at least 90%, at least 95%, or 100%. In some embodiments, the population of T cells is a heterogenous population with respect to the type of T cell.

In some embodiments, the lactate receptor overexpressing or chimeric lactate receptor expressing T cells are administered to a subject in a population of cells that includes one or more additional cells other than T cells. The additional cell can be, but is not limited to, an antigen presenting cell, a dendritic cell, a B cell, a macrophage, a neutrophil, an erythrocyte, an endothelial cell, an epithelial cell, a parenchymal cell, or a cancer cell.

Treating cancer includes, but is not limited to, reducing or inhibiting cancer cell growth, reducing or inhibiting tumor growth, reducing tumor progression, reducing tumor mass, inhibiting or reducing metastasis, reducing or inhibiting the development of metastatic cancer, and/or increasing survival or prolonging life of the subject. In some embodiments, administration of lactate receptor overexpressing or chimeric lactate receptor expressing T cells enhances T cell infiltration of the tumor.

In some embodiments, the described methods can be used to treat cancer in a human. In some embodiments, the described methods can be used to treat cancer in non-human animals or mammals. A non-human mammal can be, but is not limited to, a mouse, a rat, a rabbit, a dog, a cat, a pig, a cow, a sheep, a horse, or a non-human primate.

The term cancer includes diseases generally characterized by inappropriate cellular proliferation, or abnormal or excessive cellular proliferation. Cancers amenable to treatment using lactate receptor overexpressing or chimeric lactate receptor expressing T cells include noninvasive, invasive, superficial, papillary, flat, metastatic, localized, unicentric, multicentric, low grade, and high grade tumors. These growths may manifest themselves as any of a lesion, a polyp, a neoplasm, a papilloma, a malignancy, a sarcoma, a carcinoma, or lump, or any other type of solid tumor.

In some embodiments, cancers amenable to treatment using the described lactate receptor overexpressing or chimeric lactate receptor expressing T cells include cancers include solid tumors and cancers having a tumor or tumor cells that demonstrate high glycolysis activity, glycolytic switch, or increase glucose uptake. In some embodiments, cancers amenable to treatment using the described lactate receptor overexpressing or chimeric lactate receptor expressing T cells include cancers comprising a solid tumor having elevated lactate concentrations in the tumor or tumor microenvironment. Glycolytic activity of cancers can be used to provide information about the pathologic differentiation and staging of tumors. In some embodiments, the tumor or tumor cells exhibit increase lactate production relative to normal non-cancerous cells adjacent to the tumor. In some embodiments, the tumor or tumor cells have increased glucose metabolism relative to normal non-cancerous cells adjacent to the tumor. In some embodiments, the tumor or tumor microenvironment as decreased glucose concentration relative to normal non-cancerous tissue adjacent to the tumor. In some embodiments, the tumor or tumor microenvironment as increase lactate concentration relative to normal non-cancerous tissue adjacent to the tumor. Lactate levels in a tumor or tumor microenvironment can be measured using methods known in the art. Glucose uptake by a solid tumor can be measured using methods known in the art. Such methods include, but are not limited to, positron emission tomography (PET) with a radiolabeled analog of glucose (e.g., 18F-fluorodeoxyglucose) and magnetic resonance spectroscopy (MRS) to image the conversion of 13C-labeled pyruvate to lactate. In some embodiments, glucose uptake and metabolism by a tumor or tumor cells is determined using positron emission tomography (PET). PET imaging uses a radioisotope-labeled glucose tracer, 18F-fluorodeoxyglucose (18F-FDG), to identify areas of increase glucose uptake/metabolism in the body. The labelled-glucose analogue is transported into the cells by glucose transporters (e.g., GLUT1), and consequently phosphorylated by the hexokinase to produce 18F-FDG-6-phosphate (18F-FDG-6-p). After entering the cell, 18F-FDG-6-p is trapped and accumulates in the cytoplasm since this molecule cannot be further metabolized. Therefore, the accumulated amounts of 18F-FDG-6-p are used to identify and confirm the presence of solid tumors (showing increased glycolytic flux). Thus, the tumor amenable to treatment with the described lactate receptor overexpressing or chimeric lactate receptor expressing T cells can be, but is not limited to, a tumor that accumulates increase 18F-FDG-6-phosphate as determined by PET scan relative to nearby non-cancerous tissue.

The cancer can be, but is not limited to, HGG, pancreas, skin, brain, cervical, liver, gall bladder, stomach, lymph node, breast, lung, head and neck, larynx, pharynx, lip, throat, heart, kidney, muscle, colon, prostate, thymus, testis, uterine, ovary, cutaneous, and subcutaneous cancers. Skin cancer can be, but is not limited to, melanoma and basal cell carcinoma. Breast cancer can be, but is not limited to, ER positive breast cancer, ER negative breast cancer, and triple negative breast cancer.

### EXAMPLES

### Example 1. LacR28 Constructs

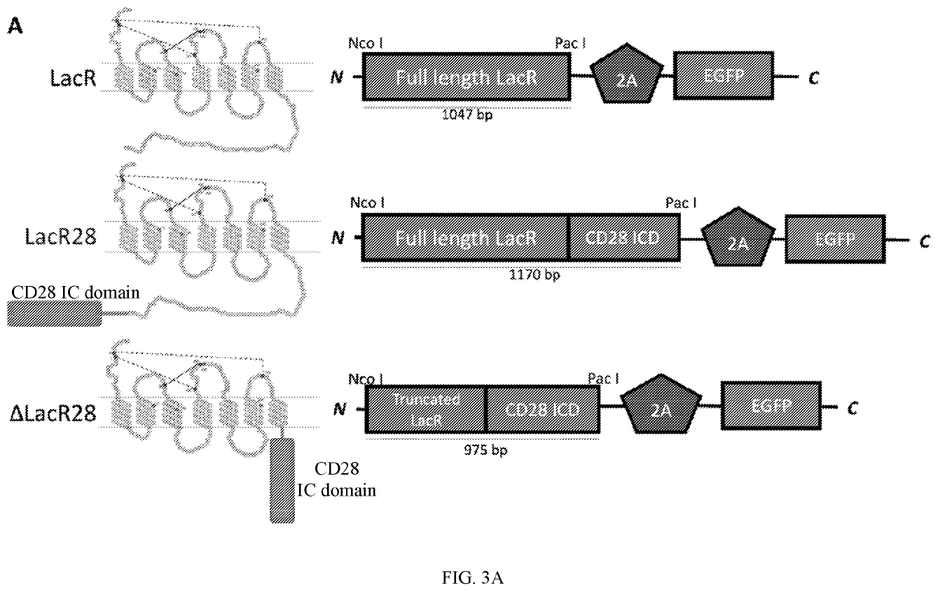

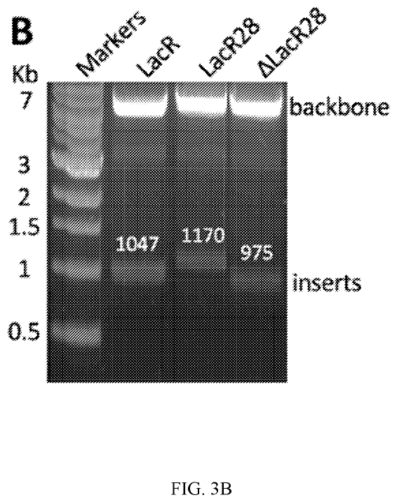

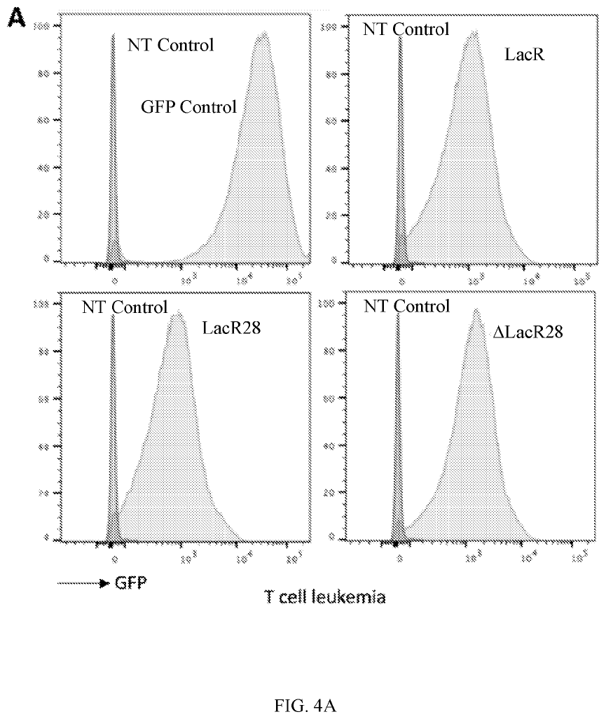

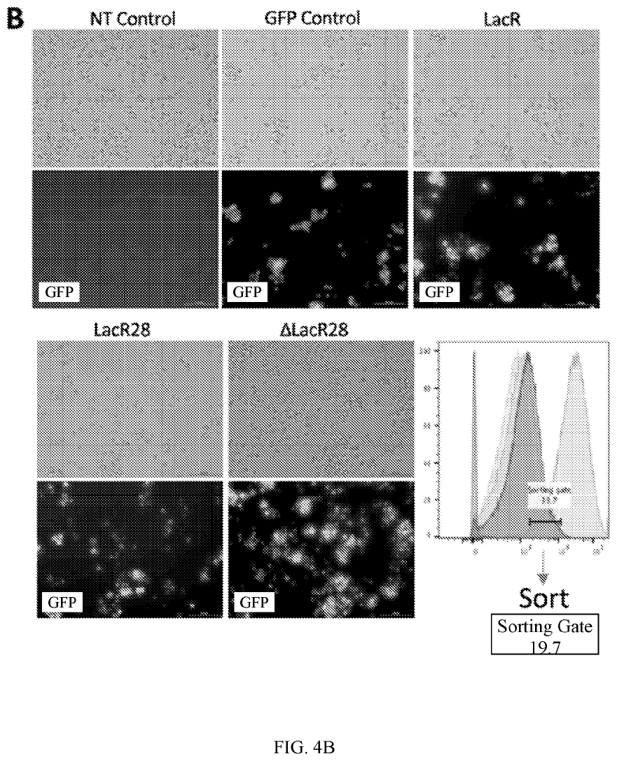

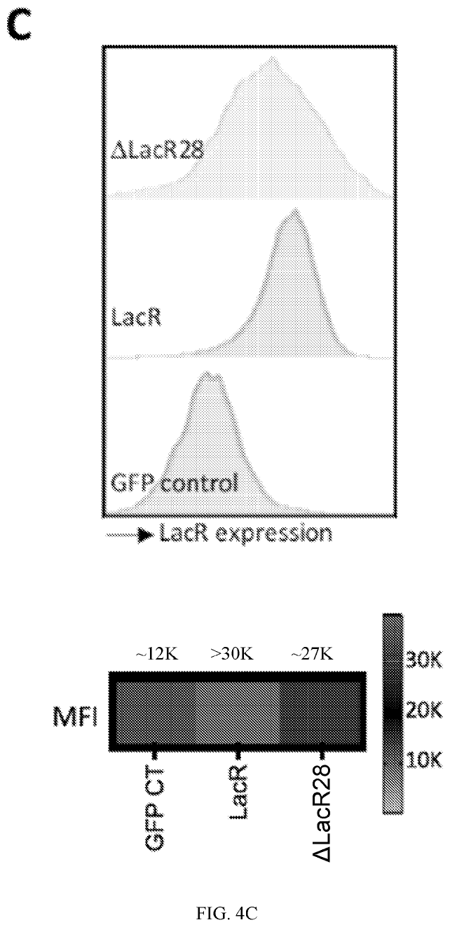

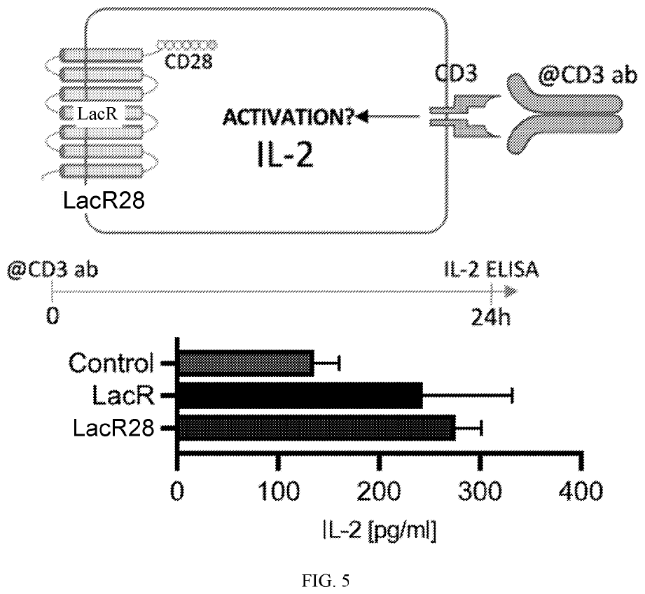

Lactate receptor (LacR), also known as Hydroxycarboxylic Acid Receptor 1 (HCAR1), is characterized by its 7-transmembrane domain structure with an N-terminus that interacts with extracellular components and a C-terminus that is responsible for transmitting intracellular signals (FIG. 3A). LacR can be expressed by immune cells such as antigen presenting cells, however its expression in T cells is poorly characterized. CD28 molecule contains a single transmembrane domain and a short cytoplasmic tail. We constructed two chimeric receptors combining (a) full length of LacR with the intracellular domain of CD28, and (b) truncated LacR (without the intracellular domain) with the intracellular domain of CD28 (FIG. 3A). pMSGV8 plasmid served as the backbone of all modifications. All cDNAs (LacR, LacR28, ΔLacR28) were synthesized by Integrated DNA Technology (IDT, California, USA) and respectively subcloned into upstream of EGFP in MSGV8 vector. 2A peptide is used to separate EGFP and upstream gene products (FIG. 3A). Enzymatic digestion at the Ncol and Pacl sites confirmed correct insertion of the respective sequences (FIG. 3B). VSV-G-pseudotyped viral particles encoding the different forms of wild-type or chimeric lactate receptor were produced by transient transfection of GP2-293 cells. We validated our constructs in Jurkat cells, which are immortalized human T lymphocyte cells. Jurkat cells were virally transduced to permanently express LacR, LacR28 or ΔLacR28. Transduction efficiency based on GFP expression was measured by flow cytometry (FIG. 4A) and confirmed using microscopy (FIG. 4B). Lactate receptor expression was also confirmed by flow cytometry (FIG. 4C). Cells engineered to express LacR or LacR28 remain functional as demonstrated by their ability to secrete IL-2 when activated with an anti-CD3 antibody (FIG. 5). Supernatants from Jurkat T cell cultures were collected 24 h after cell activation using anti-CD3 antibody. IL-2 concentrations measured by ELISA were compared between the different cell populations.

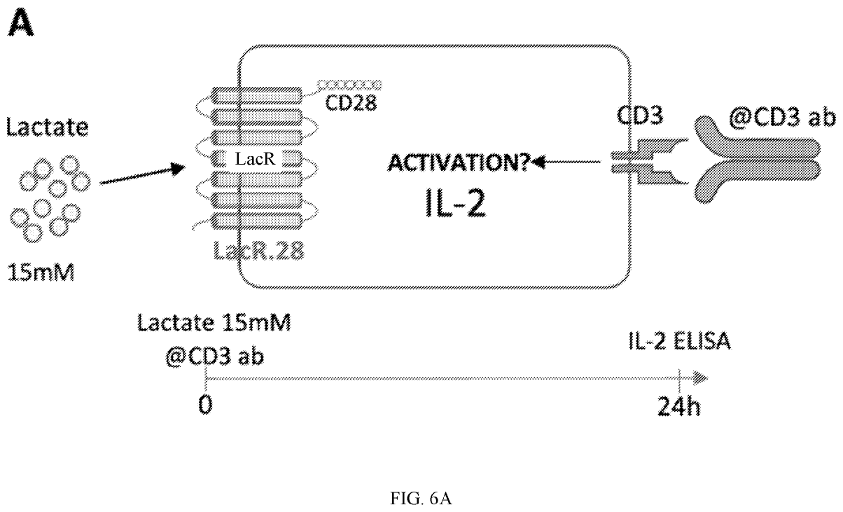

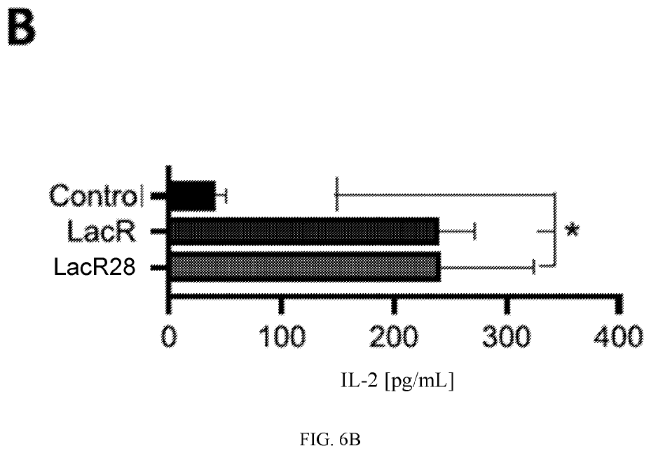

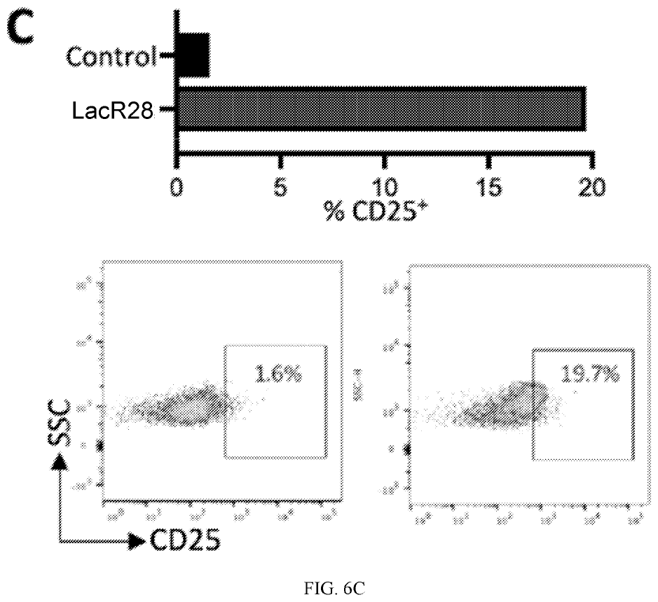

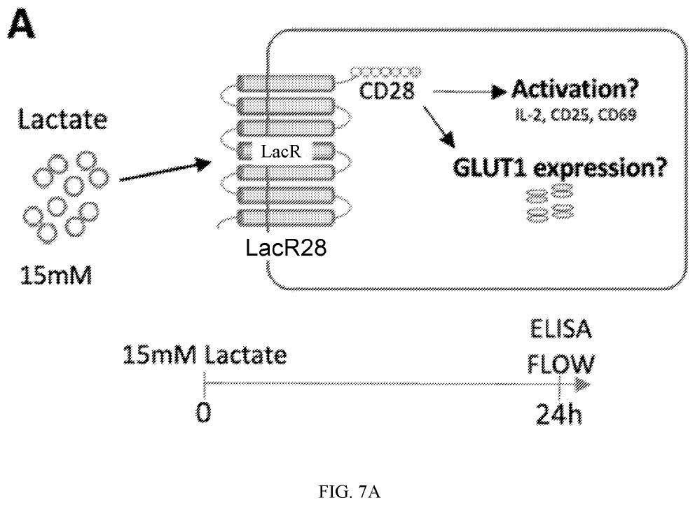

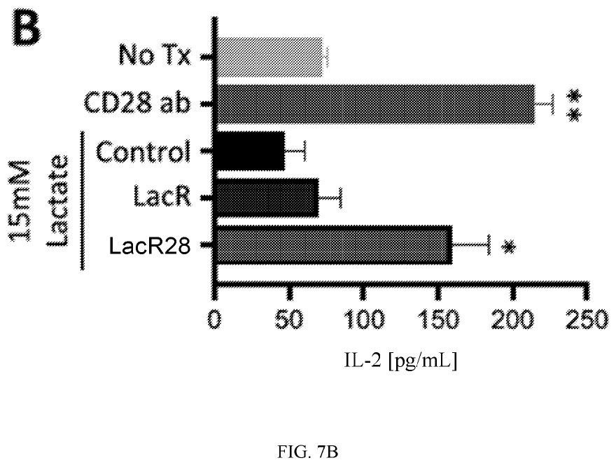

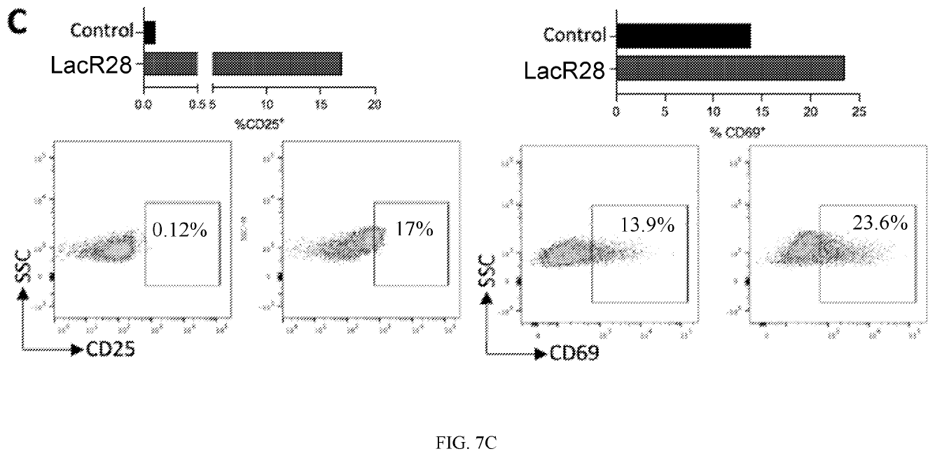

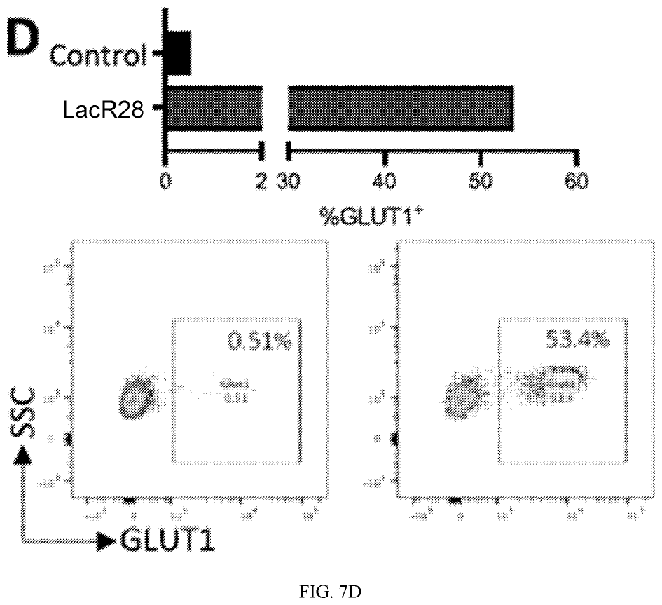

Additionally, the presence of lactate at a concentration similar to what is observed in HGG tumor microenvironment (15 mM) did not affect the CD3-induced activation of LacR and LacR28 expressing cells, but inhibited the activation of control cells (FIG. 6A-B). Supernatants from Jurkat T cell cultures were collected 24 h after treatment with anti-CD3 antibody and 15 mM of lactate. IL-2 concentrations measured by ELISA were compared between the different cell populations. IL-2 synthesis, measured 24 h after treatment with anti-CD3 and 15 mM lactate, was only reduced in control cells. We compared the expression of CD25, a marker of T cell activation, and identified a significant increase of CD25 positive cells in culture expressing LacR28 compared to control cells when stimulated with anti-CD3 and lactate (FIG. 6C). The presence of lactate significantly reduced activation of unmodified cells compared to LacR and LacR28 expressing cells. This data suggests that metabolically modified cells expressing LacR28 have the ability to overcome lactate inhibitory effect. Finally, we demonstrated the functionality of the chimeric metabolic switch receptor LacR28 by comparing the effect of lactate alone on T cell activation and GLUT1 expression (FIG. 7A). Supernatants from Jurkat T cell cultures were collected 24 h after treatment with 15 mM of lactate alone. Secretion of IL-2 measured by ELISA was significantly greater in cells expressing the chimeric receptor LacR28 compared to controls and cells expressing wild type LacR. Lactate alone activated LacR28 expressing cells, which secrete significantly higher concentration of IL-2 compared to controls or LacR cells (FIG. 7B). Lactate inhibited activation of control T cells but not in modified T cells. Increased lactate-induced activation in LacR28 cells was further supported by the up-regulation of both T cell activation markers CD25 and CD69. CD69 and CD25 were both up regulated upon T cell activation but with different kinetics. CD69 is an early activation marker whereas CD25 is up-regulated later than CD69. Both of these markers were significantly more up-regulated in LacR28 cells after 24h treatment with 15 mM lactate compared to controls (FIG. 7C). Engagement of lactate with the chimeric receptor, activating the positive CD28 signals resulted in an increased expression of GLUT1 (FIG. 7D). GLUT1 expression was higher in LacR28 cells in response to lactate treatment compared to control. These studies confirm the functionality of the chimeric metabolic switch receptor, which can be activated by lactate and results in upregulation of GLUT1 expression and T cell activation.

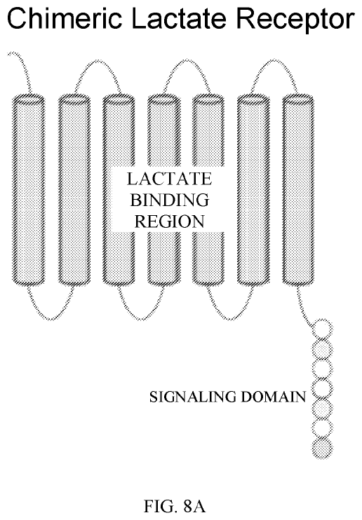

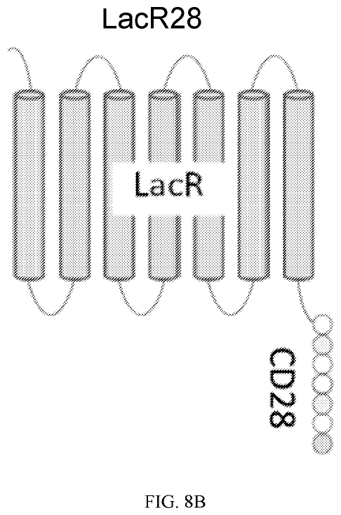

FIG. 8 illustrates an exemplary chimeric lactate receptor. The shown chimeric lactate receptor comprises a CD28 intracellular signaling domain. However, other intracellular signaling domains, including combinations of intracellular signaling domains, can readily be substituted for the CD28 intracellular signaling domain. Lactate levels typically observed in tumor microenvironments bind to the chimeric lactate receptor and generate a signal through the intracellular signaling domain. Lactate-induced signaling through the chimeric lactate receptor increases activation markers, IL-2 secretion, and GLUT1 expression in T cells expressing the chimeric lactate receptor.

### Example 2. Chimeric Lactate Receptor in Primary T Cells

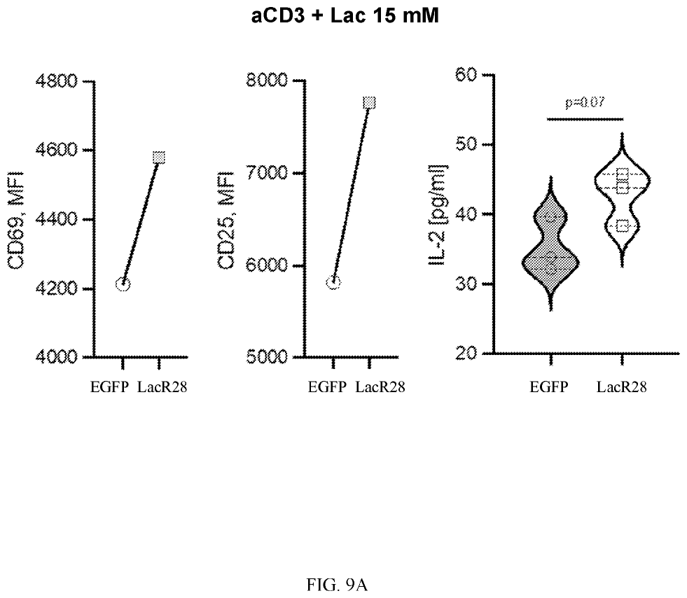

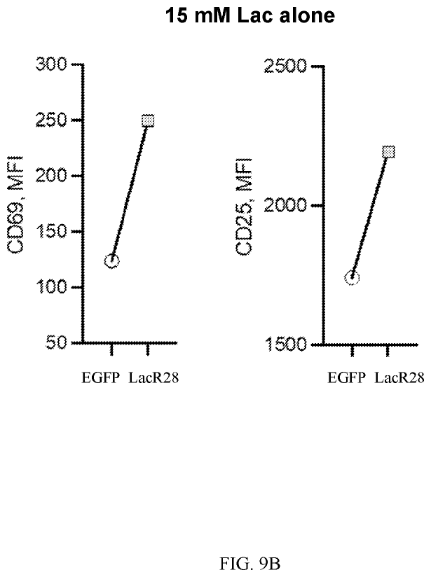

Plasmids encoding a LacR28 chimeric receptor or a control EGFP were transfected into primary T cells isolated from human donors. The transfected primary T cells were then incubated in the presence of 15 mM lactate and anti-CD3 antibody or 15 mM lactate alone (without anti-CD-3 antibody). As shown in FIG. 9A, primary T cells expressing the LacR28 chimeric receptor had greater levels of expression of CD69 and CD25, both markers of T cells activation, compared to control EGFP expressing cells. Supernatants from the primary T cell were collected after 24 h and IL-2 concentrations were measured by ELISA. As shown in FIG. 9A, primary T cells expressing the LacR28 chimeric receptor had a higher amount of IL-2 secretion, further supporting a greater level of activation compared to controls. In the presence of lactate alone As shown in FIG. 9B, increased expression of CD69 and CD25 was observed for primary T cells expressing the LacR28 chimeric receptor.

The results demonstrate the functionality of the chimeric lactate receptors in primary T cells.

1. A chimeric lactate receptor comprising a lactate receptor domain and an intracellular signaling domain.

2. The chimeric lactate receptor of claim 1, wherein the lactate receptor domain comprises LacR or ΔLacR.

3. The chimeric lactate receptor of claim 2, wherein the lactate receptor domain comprises a polypeptide having at least 70%, at least 80%, at least 90%, at least 95%, at least 97%, at least 98%, at least 99%, or 100% identity to SEQ ID NO: 2 or SEQ ID NO: 10.

4. The chimeric lactate receptor of claim 1, wherein the intracellular signaling domain comprises a primary signaling domain or a costimulatory protein signaling domain.

5. The chimeric lactate receptor of claim 1, wherein the intracellular signaling domain comprises a CD28 domain, a CD3-zeta domain, a 4-1BB domain, an OX-40 domain, a CD27 domain, a DAP10 domain, an inducible costimulatory (ICOS) domain, a 2B4 domain, an AKT domain, an IRS domain, or a PI3K domain.

6. The chimeric lactate receptor of claim 1, wherein the intracellular signaling domain comprises a CD28 domain.

7. The chimeric lactate receptor of claim 6, wherein the CD28 domain at least 80%, at least 90%, at least 95%, at least 97%, at least 98%, at least 99%, or 100% identity to SEQ ID NO: 8.

8. The chimeric lactate receptor of claim 1, wherein the lactate receptor domain comprises LacR or ΔLacR and the intracellular signaling domain comprises a CD28 domain.

9. The chimeric lactate receptor of claim 8, wherein the chimeric lactate receptor a polypeptide having at least 70%, at least 80%, at least 90%, at least 95%, at least 97%, at least 98%, at least 99%, or 100% identity to SEQ ID NO: 4 or SEQ ID NO: 6.

10. A nucleic acid encoding the chimeric lactate receptor of any one of claims 1-9.

11. The nucleic acid of claim 10, wherein nucleic acid comprises a sequence having at least 70%, at least 80%, at least 90%, at least 95%, at least 97%, at least 98%, at least 99%, or 100% identity SEQ ID NO: 1 or SEQ ID NO: 9.

12. The nucleic acid of claim 9, wherein nucleic acid comprises a sequence having at least 70%, at least 80%, at least 90%, at least 95%, at least 97%, at least 98%, at least 99%, or 100% identity SEQ ID NO: 7.

13. The nucleic acid of claim 10, wherein nucleic acid comprises a sequence having at least 70%, at least 80%, at least 90%, at least 95%, at least 97%, at least 98%, at least 99%, or 100% identity SEQ ID NO: 3 or SEQ ID NO: 5.

14. A nucleic acid vector comprising the nucleic acid sequence of any one of claims 10-13.

15. The nucleic acid vector of claim 14, wherein the nucleic acid vector comprises a DNA, an RNA, an mRNA, a double stranded nucleic acid, a single stranded nucleic acid, a plasmid, an expression vector, a viral vector, or a CRISPR construct.

16. An engineered T cell expressing the chimeric lactate receptor of any one of claims 1-9 or the nucleic acid of any one of claims 10-13.

17. The engineered T cell, wherein the engineered T cell overexpresses a lactate receptor.

18. The engineered T cell of claim 17, wherein the engineered T cell comprises a nucleic acid encoding a heterologous lactate receptor.

19. The engineered T cell of claim 18, wherein the lactate receptor comprises LacR.

20. A method of treating cancer comprising administering to a subject the T cell of any one of claims 16-19.

21. A method of treating cancer in a subject comprising:
(a) obtaining or having obtained one or more T cells from the subject or a donor subject;
(b) modifying the T cells to form lactate receptor overexpressing or chimeric lactate receptor expressing T cells; and
(c) administering the lactate receptor overexpressing or chimeric lactate receptor expressing T cells to the subject.

22. The method of claim 21, wherein modifying the T cells to form lactate receptor overexpressing or chimeric lactate receptor expressing T cells comprises inserting into the T cells the nucleic acid of any one of claims 10-13, the nucleic acid vector of claim 14 or 15, or a nucleic acid encoding a heterologous lactate receptor.

23. The method of claim 20, wherein the cancer is glioma.


SEQUENCE LISTING (10 sequences)

SEQ ID NO: 1 - DNA, 1041 bp, synthetic construct
  source 1..1041: mol_type = other DNA
atgggatacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccacggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcaaacctcggtcgcaggagttgcatcagtgtggcaaatagtttccaaagccagtctgatgggcaatgggatccccacattgttgagtggcac

SEQ ID NO: 2 - protein, 347 aa, synthetic construct
MGYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 3 - DNA, 1164 bp, synthetic construct
  source 1..1164: mol_type = other DNA
atgggatacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccacggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcaaacctcggtcgcaggagttgcatcagtgtggcaaatagtttccaaagccagtctgatgggcaatgggatccccacattgttgagtggcacaggagtaagaggagcaggctcctgcacagtgactacatgaacatgactccccgccgccccgggcccacccgcaagcattaccagccctatgccccaccacgcgacttcgcagcctatcgctcc

SEQ ID NO: 4 - protein, 388 aa, synthetic construct
MGYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWHRSKRSRLLHSDYMNMTPRRPGPTRKHYQPYAPPRDFAAYRS

SEQ ID NO: 5 - DNA, 969 bp, synthetic construct
  source 1..969: mol_type = other DNA
atgggatacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccacggggccctgcacataaccctcagcttcacctacatgaacagcatgctggatcccctggtgtattatttttcaaggagtaagaggagcaggctcctgcacagtgactacatgaacatgactccccgccgccccgggcccacccgcaagcattaccagccctatgccccaccacgcgacttcgcagcctatcgctcc

SEQ ID NO: 6 - protein, 323 aa, synthetic construct
MGYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSRSKRSRLLHSDYMNMTPRRPGPTRKHYQPYAPPRDFAAYRS

SEQ ID NO: 7 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 8 - protein, 41 aa, synthetic construct
RSKRSRLLHSDYMNMTPRRPGPTRKHYQPYAPPRDFAAYRS

SEQ ID NO: 9 - DNA, 846 bp, synthetic construct
  source 1..846: mol_type = other DNA
atgggatacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccacggggccctgcacataaccctcagcttcacctacatgaacagcatgctggatcccctggtgtattatttttca

SEQ ID NO: 10 - protein, 282 aa, synthetic construct
MGYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFS